SEQUENCE LISTING (31 sequences)

SEQ ID NO: 1 - DNA, 20 bp, Artificial Sequence
  Synthetic oligonucleotide
tgaaggtgagctggagccat

SEQ ID NO: 2 - DNA, 20 bp, Artificial Sequence
  Synthetic oligonucleotide
gctccattgatgcagcttaa

SEQ ID NO: 3 - DNA, 20 bp, Artificial Sequence
  Synthetic oligonucleotide
ccctgtatgtgctccattga

SEQ ID NO: 4 - DNA, 20 bp, Artificial Sequence
  Synthetic oligonucleotide
ggtgcaaagtcaactagaag

SEQ ID NO: 5 - DNA, 20 bp, Artificial Sequence
  Synthetic oligonucleotide
attcttaaacctgagggagc

SEQ ID NO: 6 - DNA, 20 bp, Artificial Sequence
  Synthetic oligonucleotide
gatgcagcttaaacaattct

SEQ ID NO: 7 - DNA, 20 bp, Artificial Sequence
  Synthetic oligonucleotide
cagcactgcaaatggcttcc

SEQ ID NO: 8 - DNA, 20 bp, Artificial Sequence
  Synthetic oligonucleotide
tcccgcctgtgacatgcatt

SEQ ID NO: 9 - DNA, 20 bp, Artificial Sequence
  Synthetic oligonucleotide
gccgagtgccttgcctggaa

SEQ ID NO: 10 - DNA, 20 bp, Artificial Sequence
  Synthetic oligonucleotide
agagatgcagctggagccat

SEQ ID NO: 11 - DNA, 20 bp, Artificial Sequence
  Synthetic oligonucleotide
aggtgaaggcctggagccat

SEQ ID NO: 12 - DNA, 20 bp, Artificial Sequence
  Synthetic oligonucleotide
gtctggcgctgcaccactct

SEQ ID NO: 13 - DNA, 20 bp, Artificial Sequence
  Synthetic oligonucleotide
ctgatttccaaaatcccatg

SEQ ID NO: 14 - DNA, 20 bp, Artificial Sequence
  Synthetic oligonucleotide
ctgggctgtttggtgcctta

SEQ ID NO: 15 - DNA, 20 bp, Artificial Sequence
  Synthetic oligonucleotide
tcagggctggactgcctgct

SEQ ID NO: 16 - DNA, 20 bp, Artificial Sequence
  Synthetic oligonucleotide
ggtgagggagcgggaggcgg

SEQ ID NO: 17 - DNA, 20 bp, Artificial Sequence
  Synthetic oligonucleotide
cgctcctcctccccgcggcg

SEQ ID NO: 18 - DNA, 20 bp, Artificial Sequence
  Synthetic oligonucleotide
ttcggcggcagcttctcgcc

SEQ ID NO: 19 - DNA, 20 bp, Artificial Sequence
  Synthetic oligonucleotide
gccgccccaacgtcctgtcg

SEQ ID NO: 20 - DNA, 20 bp, Artificial Sequence
  Synthetic oligonucleotide
tcctcctccccgcggcgggt

SEQ ID NO: 21 - DNA, 20 bp, Artificial Sequence
  Synthetic oligonucleotide
ctcgcccgctcctcctcccc

SEQ ID NO: 22 - DNA, 20 bp, Artificial Sequence
  Synthetic oligonucleotide
ctggcttctcctcctcccct

SEQ ID NO: 23 - DNA, 20 bp, Artificial Sequence
  Synthetic oligonucleotide
cgggaggcggtcacattcgg

SEQ ID NO: 24 - DNA, 20 bp, Artificial Sequence
  Synthetic oligonucleotide
tctggcgctgcaccactctc

SEQ ID NO: 25 - DNA, 20 bp, Artificial Sequence
  Synthetic oligonucleotide
ttctcgcccgctcctcctcc

SEQ ID NO: 26 - DNA, 20 bp, Artificial Sequence
  Synthetic oligonucleotide
ttctcctcctcccctggcag

SEQ ID NO: 27 - DNA, 20 bp, Artificial Sequence
  Synthetic oligonucleotide
cctgctggcttctcctcctc

SEQ ID NO: 28 - DNA, 20 bp, Artificial Sequence
  Synthetic oligonucleotide
ucccgcctgtgacaugcauu

SEQ ID NO: 29 - DNA, 3291 bp, Homo sapiens
agaatcggagagccggtggcgtcgcaggtcgggaggacgagcaccgagtcgagggctcgctcgtctgggccgcccgagagtcttaatcgcgggcgcttgggccgccatcttagatggcgggagtaagaggaaaacgattgtgaggcgggaacggctttctgctgccttttttgggccccgaaaagggtcagctggccgggctttggggcgcgtgccctgaggcgcggagcgcgtttgctacgatgcgggggctgctcggggctccgtcccctgggctggggacgcgccgaatgtgaccgcctcccgctccctcacccgccgcggggaggaggagcgggcgagaagctgccgccgaacgacaggacgttggggcggcctggctccctcaggtttaagaattgtttaagctgcatcaatggagcacatacagggagcttggaagacgatcagcaatggttttggattcaaagatgccgtgtttgatggctccagctgcatctctcctacaatagttcagcagtttggctatcagcgccgggcatcagatgatggcaaactcacagatccttctaagacaagcaacactatccgtgttttcttgccgaacaagcaaagaacagtggtcaatgtgcgaaatggaatgagcttgcatgactgccttatgaaagcactcaaggtgaggggcctgcaaccagagtgctgtgcagtgttcagacttctccacgaacacaaaggtaaaaaagcacgcttagattggaatactgatgctgcgtctttgattggagaagaacttcaagtagatttcctggatcatgttcccctcacaacacacaactttgctcggaagacgttcctgaagcttgccttctgtgacatctgtcagaaattcctgctcaatggatttcgatgtcagacttgtggctacaaatttcatgagcactgtagcaccaaagtacctactatgtgtgtggactggagtaacatcagacaactcttattgtttccaaattccactattggtgatagtggagtcccagcactaccttctttgactatgcgtcgtatgcgagagtctgtttccaggatgcctgttagttctcagcacagatattctacacctcacgccttcacctttaacacctccagtccctcatctgaaggttccctctcccagaggcagaggtcgacatccacacctaatgtccacatggtcagcaccaccctgcctgtggacagcaggatgattgaggatgcaattcgaagtcacagcgaatcagcctcaccttcagccctgtccagtagccccaacaatctgagcccaacaggctggtcacagccgaaaacccccgtgccagcacaaagagagcgggcaccagtatctgggacccaggagaaaaacaaaattaggcctcgtggacagagagattcaagctattattgggaaatagaagccagtgaagtgatgctgtccactcggattgggtcaggctcttttggaactgtttataagggtaaatggcacggagatgttgcagtaaagatcctaaaggttgtcgacccaaccccagagcaattccaggccttcaggaatgaggtggctgttctgcgcaaaacacggcatgtgaacattctgcttttcatggggtacatgacaaaggacaacctggcaattgtgacccagtggtgcgagggcagcagcctctacaaacacctgcatgtccaggagaccaagtttcagatgttccagctaattgacattgcccggcagacggctcagggaatggactatttgcatgcaaagaacatcatccatagagacatgaaatccaacaatatatttctccatgaaggcttaacagtgaaaattggagattttggtttggcaacagtaaagtcacgctggagtggttctcagcaggttgaacaacctactggctctgtcctctggatggccccagaggtgatccgaatgcaggataacaacccattcagtttccagtcggatgtctactcctatggcatcgtattgtatgaactgatgacgggggagcttccttattctcacatcaacaaccgagatcagatcatcttcatggtgggccgaggatatgcctccccagatcttagtaagctatataagaactgccccaaagcaatgaagaggctggtagctgactgtgtgaagaaagtaaaggaagagaggcctctttttccccagatcctgtcttccattgagctgctccaacactctctaccgaagatcaaccggagcgcttccgagccatccttgcatcgggcagcccacactgaggatatcaatgcttgcacgctgaccacgtccccgaggctgcctgtcttctagttgactttgcacctgtcttcaggctgccaggggaggaggagaagccagcaggcaccacttttctgctccctttctccagaggcagaacacatgttttcagagaagctgctgctaaggaccttctagactgctcacagggccttaacttcatgttgccttcttttctatccctttgggccctgggagaaggaagccatttgcagtgctggtgtgtcctgctccctccccacattccccatgctcaaggcccagccttctgtagatgcgcaagtggatgttgatggtagtacaaaaagcaggggcccagccccagctgttggctacatgagtatttagaggaagtaaggtagcaggcagtccagccctgatgtggagacacatgggattttggaaatcagcttctggaggaatgcatgtcacaggcgggactttcttcagagagtggtgcagcgccagacattttgcacataaggcaccaaacagcccaggactgccgagactctggccgcccgaaggagcctgctttggtactatggaacttttcttaggggacacgtcctcctttcacagcttctaaggtgtccagtgcattgggatggttttccaggcaaggcactcggccaatccgcatctcagccctctcagggagcagtcttccatcatgctgaattttgtcttccaggagctgcccctatggggcggggccgcagggccagccttgtttctctaacaaacaaacaaacaaacagccttgtttctctagtcacatcatgtgtatacaaggaagccaggaatacaggttttcttgatgatttgggttttaattttgtttttattgcacctgacaaaatacagttatctgatggtccctcaattatgttattttaataaaataaattaaatttaggtgtaaaaaaaaaaaaaaaaaa

SEQ ID NO: 30 - DNA, 2503 bp, Homo sapiens
tgacccaataagggtggaaggctgagtcccgcagagccaataacgagagtccgagaggcgacggaggcggactctgtgaggaaacaagaagagaggcccaagatggagacggcggcggctgtagcggcgtgacaggagccccatggcacctgcccagccccacctcagcccatcttgacaaaatctaaggctccatggagccaccacggggcccccctgccaatggggccgagccatcccgggcagtgggcaccgtcaaagtatacctgcccaacaagcaacgcacggtggtgactgtccgggatggcatgagtgtctacgactctctagacaaggccctgaaggtgcggggtctaaatcaggactgctgtgtggtctaccgactcatcaagggacgaaagacggtcactgcctgggacacagccattgctcccctggatggcgaggagctcattgtcgaggtccttgaagatgtcccgctgaccatgcacaattttgtacggaagaccttcttcagcctggcgttctgtgacttctgccttaagtttctgttccatggcttccgttgccaaacctgtggctacaagttccaccagcattgttcctccaaggtccccacagtctgtgttgacatgagtaccaaccgccaacagttctaccacagtgtccaggatttgtccggaggctccagacagcatgaggctccctcgaaccgccccctgaatgagttgctaaccccccagggtcccagcccccgcacccagcactgtgacccggagcacttccccttccctgccccagccaatgcccccctacagcgcatccgctccacgtccactcccaacgtccatatggtcagcaccacggcccccatggactccaacctcatccagctcactggccagagtttcagcactgatgctgccggtagtagaggaggtagtgatggaaccccccgggggagccccagcccagccagcgtgtcctcggggaggaagtccccacattccaagtcaccagcagagcagcgcgagcggaagtccttggccgatgacaagaagaaagtgaagaacctggggtaccgggactcaggctattactgggaggtaccacccagtgaggtgcagctgctgaagaggatcgggacgggctcgtttggcaccgtgtttcgagggcggtggcatggcgatgtggccgtgaaggtgctcaaggtgtcccagcccacagctgagcaggcccaggctttcaagaatgagatgcaggtgctcaggaagacgcgacatgtcaacatcttgctgtttatgggcttcatgacccggccgggatttgccatcatcacacagtggtgtgagggctccagcctctaccatcacctgcatgtggccgacacacgcttcgacatggtccagctcatcgacgtggcccggcagactgcccagggcatggactacctccatgccaagaacatcatccaccgagatctcaagtctaacaacatcttcctacatgaggggctcacggtgaagatcggtgactttggcttggccacagtgaagactcgatggagcggggcccagcccttggagcagccctcaggatctgtgctgtggatggcagctgaggtgatccgtatgcaggacccgaacccctacagcttccagtcagacgtctatgcctacggggttgtgctctacgagcttatgactggctcactgccttacagccacattggctgccgtgaccagattatctttatggtgggccgtggctatctgtccccggacctcagcaaaatctccagcaactgccccaaggccatgcggcgcctgctgtctgactgcctcaagttccagcgggaggagcggcccctcttcccccagatcctggccacaattgagctgctgcaacggtcactccccaagattgagcggagtgcctcggaaccctccttgcaccgcacccaggccgatgagttgcctgcctgcctactcagcgcagcccgccttgtgccttaggccccgcccaagccaccagggagccaatctcagccctccacgccaaggagccttgcccaccagccaatcaatgttcgtctctgccctgatgctgcctcaggatcccccattccccaccctgggagatgagggggtccccatgtgcttttccagttcttctggaattgggggacccccgccaaagactgagccccctgtctcctccatcatttggtttcctcttggctttggggatacttctaaattttgggagctcctccatctccaatggctgggatttgtggcagggattccactcagaacctctctggaatttgtgcctgatgtgccttccactggattttggggttcccagcaccccatgtggattttggggggtcccttttgtgtctcccccgccattcaaggactcctctctttcttcaccaagaagcacagaattctgctgggcctttgcttgtttaaaaaaaaaaaaaaaaaaaaaaaaa

SEQ ID NO: 31 - DNA, 2949 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

(19) United States
(12) Patent Application Publication
(10) Pub. No.: US 2013/0028889 A1
(43) Pub. Date: Jan. 31, 2013

(54) DOSING REGIMENS FOR TREATING AND PREVENTING OCULAR DISORDERS USING C-RAF ANTISENSE

(71) Applicants: Peter Hnik, Vancouver (CA); John G. Clement, Delta (CA)

(72) Inventors: Peter Hnik, Vancouver (CA); John G. Clement, Delta (CA)

(73) Assignee: ICO THERAPEUTICS INC., Vancouver, BC (CA)

(21) Appl. No.: 13/577,219

(22) PCT Filed: Feb. 3, 2011
(86) PCT No.: PCT/US11/23642

§ 371 Date: Oct. 17, 2012

Related U.S. Application Data
(60) Provisional application No. 61/301,499, filed on Feb. 4, 2010.

(51) Int. Cl. A61K 31/7052; A61P 27/02; A61K 39/395

(52) U.S. Cl. 424/133.1; 514/44 A; 424/145.1

(57) ABSTRACT

The present invention provides methods and dosing regimens for treating and preventing ocular disorders using c-raf antisense oligonucleotides, alone or in combination with other agents.

### CROSS-REFERENCE TO RELATED APPLICATION

This application claims the benefit under 35 U.S.C. §119(e) of U.S. Provisional Patent Application No. 61/301,499 filed Feb. 4, 2010, where this provisional application is incorporated herein by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 480231_{—}408PC_SEQUENCE_LISTING.txt. The text file is 16 KB, was created on Feb. 3, 2011 and is being submitted electronically via EFS-Web.

### BACKGROUND

1. Technical Field

This invention relates to methods and compositions for treating and preventing ocular diseases and disorders using raf antisense oligonucleotides, including dosing regimens and unit dosage forms.

2. Description of the Related Art

The raf gene family includes three highly conserved genes termed A-raf, B-raf and c-raf (also called raf-1). Raf genes encode protein kinases that play important regulatory roles in signal transduction processes that regulate cell proliferation and angiogenesis. Antisense oligonucleotides targeting raf genes have been shown to inhibit raf gene expression and cell growth. In addition, antisense oligonucleotides targeted to raf were also shown to reduce ocular neovascularization in a pig branch retinal vein occlusion model, supporting the use of these oligonucleotides to treat undesired angiogenesis or neovascularization in the eye and other tissues and organs (U.S. Pat. No. 6,410,518). Aberrant cell growth and/or angiogenesis has been associated with a variety of ocular diseases and disorders, including, but not limited to, macular edema, macular degeneration, diabetic retinopathy, and retinopathy of prematurity.

Unfortunately, the anatomical and physiological characteristics of the eye render retinal drug delivery a major challenge. Due to the presence of a formidable blood-retinal-barrier to solute transport, high doses of drugs are required to deliver therapeutic quantities of drug to the retina following systemic administration. Such large doses can lead to severe systemic side effects. Intravitreal injections are routinely used to circumvent the blood-retinal-barrier and to better deliver drugs to the retina. While this mode of administration provides significant drug concentrations in the retina with low doses, several retinal disorders require repeated intravitreal injections, which can result in retinal detachment, vitreal hemorrhage, and cataracts. Intravitreal implants capable of delivering drugs over a few months are currently available. However, these implants require surgical placement and removal, and the use of these implants has been associated with retinal detachment and endophthalmitis.

Clearly, there remains a long-felt need for improved compositions and methods for treating and preventing ocular diseases and disorders, as well as improved methods for delivering ocular drugs to the retina.

### BRIEF SUMMARY

The present invention provides methods and compositions useful in treating and preventing ocular disorders, including dosing regimens of raf antisense oligonucleotides.

In one embodiment, the present invention provides a method of treating or preventing macular edema, comprising administering to a subject in need thereof a therapeutically effective amount of a composition comprising a modified oligonucleotide comprising or consisting of a sequence 100% complementary to nucleobases 2771 to 2790 of SEQ ID NO:29, wherein the composition is administered no more than once every 90 days. In one embodiment, the modified oligonucleotide comprises or consists of the nucleobase sequence of SEQ ID NO:28. In certain embodiments, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment; and wherein each nucleoside of each wing segment comprises a modified sugar. In particular embodiments, the modified oligonucleotide consists of 20 nucleobases. In certain embodiments, the modified oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; and wherein each cytosine is a 5-methylcytosine. In certain embodiments, the oligonucleotide comprises or consists of the sequence, UCCCGCCTGTGACAUGCAUU (SEQ ID NO:28). In particular embodiments, the oligonucleotide is a full phosphorothioate analog, wherein the oligonucleotide comprises 2′-O-methoxyethyl substitutions at positions 1-6 and 15-20, and wherein residues 7-14 are unmodified 2′-deoxy. In certain embodiments, the oligonucleotide is administered no more than once every 180 days.

In a related embodiment, the present invention provides a method of reducing or preventing excess retinal thickness, comprising administering to a subject in need thereof a therapeutically effective amount of a composition comprising a modified oligonucleotide comprising or consisting of a sequence 100% complementary to nucleobases 2771 to 2790 of SEQ ID NO:29, wherein the composition is administered no more than once every 90 days. In one embodiment, the modified oligonucleotide comprises or consists of the nucleobase sequence of SEQ ID NO:28. In certain embodiments, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment, and wherein each nucleoside of each wing segment comprises a modified sugar. In particular embodiments, the modified oligonucleotide consists of 20 nucleobases. In certain embodiments, the modified oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; and wherein each cytosine is a 5-methylcytosine. In certain embodiments, the oligonucleotide comprises or consists of the sequence, UCCCGCCTGTGACAUGCAUU (SEQ ID NO:28). In particular embodiments, the oligonucleotide is a full phosphorothioate analog, wherein the oligonucleotide comprises 2′-O-methoxyethyl substitutions at positions 1-6 and 15-20, and wherein residues 7-14 are unmodified 2′-deoxy. In certain embodiments, the oligonucleotide is administered no more than once every 180 days. In particular embodiments, the retinal thickness is reduced by at least 100 microns or by at least 300 microns.

In another related embodiment, the present invention provides a method of improving visual acuity, comprising administering to a subject in need thereof a therapeutically effective amount of a composition comprising a modified oligonucleotide comprising or consisting of a sequence 100% complementary to nucleobases 2771 to 2790 of SEQ ID NO:29, wherein the composition is administered no more than once every 90 days. In one embodiment, the modified oligonucleotide comprises or consists of the nucleobase sequence of SEQ ID NO:28. In certain embodiments, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment; and wherein each nucleoside of each wing segment comprises a modified sugar. In particular embodiments, the modified oligonucleotide consists of 20 nucleobases. In certain embodiments, the modified oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; and wherein each cytosine is a 5-methylcytosine. In certain embodiments, the oligonucleotide comprises or consists of the sequence, UCCCGCCTGTGACAUGCAUU (SEQ ID NO:28). In particular embodiments, the oligonucleotide is a full phosphorothioate analog, wherein the oligonucleotide comprises 2′-O-methoxyethyl substitutions at positions 1-6 and 15-20, and wherein residues 7-14 are unmodified 2′-deoxy. In certain embodiments, the oligonucleotide is administered no more than once every 180 days. In particular embodiments, visual acuity is improved by at least 25%.

In a further related embodiment, the present invention provides a method of treating or preventing macular edema, comprising administering to a subject in need thereof a therapeutically effective amount of a composition comprising a modified oligonucleotide comprising or consisting of a sequence 100% complementary to nucleobases 2771 to 2790 of SEQ ID NO:29, wherein at least 100 μg of the oligonucleotide is administered. In one embodiment, the modified oligonucleotide comprises or consists of the nucleobase sequence of SEQ ID NO:28. In certain embodiments, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment, and wherein each nucleoside of each wing segment comprises a modified sugar. In particular embodiments, the modified oligonucleotide consists of 20 nucleobases. In certain embodiments, the modified oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; and wherein each cytosine is a 5-methylcytosine. In certain embodiments, the oligonucleotide comprises or consists of the sequence, UCCCGCCTGTGACAUGCAUU (SEQ ID NO:28). In particular embodiments, the oligonucleotide is a full phosphorothioate analog, wherein the oligonucleotide comprises 2′-O-methoxyethyl substitutions at positions 1-6 and 15-20, and wherein residues 7-14 are unmodified 2′-deoxy. In particular embodiments, the oligonucleotide is administered no more than once every 90 days or no more than once every 180 days.

In another embodiment, the present invention includes a method of treating or preventing macular edema, wherein said method comprises administering to a subject: (1) a first composition comprising a modified oligonucleotide comprising or consisting of a sequence 100% complementary to nucleobases 2771 to 2790 of SEQ ID NO:29 and (2) a second composition comprising one or more of ranibizumab, bevacizumab, rapamycin, pegaptanib, triamcinolone, fluocinolone acetonide, and RETAANE® (anecortave acetate), wherein the first composition is administered no more than once every 90 days. In one embodiment, the modified oligonucleotide comprises or consists of the nucleobase sequence of SEQ ID NO:28. In certain embodiments, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment; and wherein each nucleoside of each wing segment comprises a modified sugar. In particular embodiments, the modified oligonucleotide consists of 20 nucleobases. In certain embodiments, the modified oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; and wherein each cytosine is a 5-methylcytosine. In certain embodiments, the oligonucleotide comprises or consists of the sequence, UCCCGCCTGTGACAUGCAUU (SEQ ID NO:28). In particular embodiments, the oligonucleotide is a full phosphorothioate analog, wherein the oligonucleotide comprises 2′-O-methoxyethyl substitutions at positions 1-6 and 15-20, and wherein residues 7-14 are unmodified 2′-deoxy. In certain embodiments, the oligonucleotide is administered no more than once every 180 days. In one embodiment, between 100 μg and 1000 μg or between 100 μg and 1500 μg of the oligonucleotide is administered. In one embodiment, between 0.1 mg and 2.0 mg of bevacizumab is administered. In one embodiment, between 0.1 mg and 2.0 mg of ranibizumab is administered. In one embodiment, between 0.1 mg and 2.0 mg of rapamycin is administered. In particular embodiments, the ranibizumab, bevacizumab, or rapamycin is administered once every about thirty days. In certain embodiments, the ranibizumab, bevacizumab, or rapamycin is administered for no more than 90 days. In particular embodiments, an effective amount of the first composition and an effective amount of the second composition are administered. In particular embodiments, the effective amount of each composition is an effective amount when administered concomitantly.

In certain embodiments of the various methods of the present invention described above and herein, an amount of at least 100 μg of the oligonucleotide is administered. In certain embodiments, an amount of between 100 μg and 1500 μg of the oligonucleotide is administered. In certain embodiments, an amount of between 100 μg and 1000 μg of the oligonucleotide is administered. In particular embodiments, the amount of oligonucleotide is administered in a dose of a pharmaceutical composition comprising the oligonucleotide. In particular embodiments of the methods of the present invention, two or more doses are administered, wherein each dose is administered at least 90 days apart.

In certain embodiments of methods of the present invention, the therapeutically effective amount of the composition comprising the oligonucleotide is administered in two or more doses, wherein each dose is administered at least 90 days apart from the other doses. In particular embodiments of methods of the present invention, a therapeutically effective amount of the oligonucleotide is administered in two or more doses of a pharmaceutical composition comprising the oligonucleotide, wherein each of the two or more doses is administered at least 90 days apart from the other doses. In other related embodiments, the composition is administered in two or more doses, each dose comprising a therapeutically effective amount of the composition, wherein the two or more doses are administered at least 90 days apart from each other.

In certain embodiments of the various methods of the present invention, the half life of the administered oligonucleotide is at least 44 days. In particular embodiments, the half-life is determined in an ocular tissue selected from retina and choroid.

In particular embodiments of the various methods of the present invention, the oligonucleotide, or composition comprising the oligonucleotide, is administered by intravitreal injection.

In certain embodiments of the various methods of the present invention, the duration of action of the oligonucleotide is at least 50 days or at least 100 days.

In certain embodiments of any of the methods of the present invention, the subject is refractory to treatment with one or more of: a steroid, laser treatment, ranibizumab, bevacizumab, and rapamycin.

The present invention further includes a modified oligonucleotide comprising or consisting of a sequence 100% complementary to nucleobases 2771 to 2790 of SEQ ID NO:29 for use, or prepared for use, in the treatment or prevention of a macular edema in a subject by administration of a therapeutically effective amount of the modified oligonucleotide to the subject no more than once every 90 days.

In a related embodiment, the present invention includes a modified oligonucleotide comprising or consisting of a sequence 100% complementary to nucleobases 2771 to 2790 of SEQ ID NO:29 for use, or prepared for use, in reducing or preventing excess retinal thickness in a subject by administration of a therapeutically effective amount of the modified oligonucleotide to the subject no more than once every 90 days.

In another related embodiment, the present invention includes a modified oligonucleotide comprising or consisting of a sequence 100% complementary to nucleobases 2771 to 2790 of SEQ ID NO:29 for use, or prepared for use, in improving visual acuity in a subject by administration of a therapeutically effective amount of the modified oligonucleotide to the subject no more than once every 90 days.

In a further related embodiment, the present invention provides a modified oligonucleotide comprising or consisting of a sequence 100% complementary to nucleobases 2771 to 2790 of SEQ ID NO:29 for use, or prepared for use, in treating or preventing macular edema in a subject by administration of at least 100 μg of the modified oligonucleotide to the subject.

In another embodiment, the present invention includes a modified oligonucleotide comprising or consisting of a sequence 100% complementary to nucleobases 2771 to 2790 of SEQ ID NO:29 for use, or prepared for use, in treating or preventing macular edema in a subject by administration of the modified oligonucleotide in combination with and one or more of ranibizumab, bevacizumab, rapamycin, pegaptanib, triamcinolone, fluocinolone acetonide, and RETAANE® (anecortave acetate), wherein the modified oligonucleotide is administered to the subject no more than once every 90 days.

The present invention further includes use of a modified oligonucleotide comprising or consisting of a sequence 100% complementary to nucleobases 2771 to 2790 of SEQ ID NO:29 for the treatment or prevention of a macular edema in a subject by administration of a therapeutically effective amount of the modified oligonucleotide to the subject no more than once every 90 days.

In a related embodiment, the present invention includes use of a modified oligonucleotide comprising or consisting of a sequence 100% complementary to nucleobases 2771 to 2790 of SEQ ID NO:29 for reducing or preventing excess retinal thickness in a subject by administration of a therapeutically effective amount of the modified oligonucleotide to the subject no more than once every 90 days.

In another related embodiment, the present invention includes use of a modified oligonucleotide comprising or consisting of a sequence 100% complementary to nucleobases 2771 to 2790 of SEQ ID NO:29 for improving visual acuity in a subject by administration of a therapeutically effective amount of the modified oligonucleotide to the subject no more than once every 90 days.

In a further related embodiment, the present invention includes use of a modified oligonucleotide comprising or consisting of a sequence 100% complementary to nucleobases 2771 to 2790 of SEQ ID NO:29 for treating or preventing macular edema in a subject by administration of at least 100 μg of the modified oligonucleotide to the subject.

In another embodiment, the present invention includes use of a modified oligonucleotide comprising or consisting of a sequence 100% complementary to nucleobases 2771 to 2790 of SEQ ID NO:29 for treating or preventing macular edema in a subject by administration of the modified oligonucleotide in combination with and one or more of ranibizumab, bevacizumab, rapamycin, pegaptanib, triamcinolone, fluocinolone acetonide, and RETAANE® (anecortave acetate), wherein the modified oligonucleotide is administered to the subject no more than once every 90 days.

The present invention further provides a modified oligonucleotide 100% complementary to nucleobases 2771 to 2790 of SEQ ID NO:29, for use in the manufacture of a medicament for the treatment of a macular edema in a subject by administration of a therapeutically effective amount of the modified oligonucleotide to the subject no more than once every 90 days.

In a related embodiment, the present invention includes a modified oligonucleotide comprising or consisting of a sequence 100% complementary to nucleobases 2771 to 2790 of SEQ ID NO:29 for use in the manufacture of a medicament for reducing or preventing excess retinal thickness in a subject by administration of a therapeutically effective amount of the modified oligonucleotide to the subject no more than once every 90 days.

In another related embodiment, the present invention includes a modified oligonucleotide comprising or consisting of a sequence 100% complementary to nucleobases 2771 to 2790 of SEQ ID NO:29 for use in the manufacture of a medicament for improving visual acuity in a subject by administration of a therapeutically effective amount of the modified oligonucleotide to the subject no more than once every 90 days.

In a further related embodiment, the present invention provides a modified oligonucleotide comprising or consisting of a sequence 100% complementary to nucleobases 2771 to 2790 of SEQ ID NO:29 for use in the manufacture of a medicament for treating or preventing macular edema in a subject by administration of at least 100 μg of the modified oligonucleotide to the subject.

In another embodiment, the present invention includes a modified oligonucleotide comprising or consisting of a sequence 100% complementary to nucleobases 2771 to 2790 of SEQ ID NO:29 for use in the manufacture of a medicament for treating or preventing macular edema in a subject by administration of the modified oligonucleotide in combination with and one or more of ranibizumab, bevacizumab, rapamycin, pegaptanib, triamcinolone, fluocinolone acetonide, and RETAANE® (anecortave acetate), wherein the modified oligonucleotide is administered to the subject no more than once every 90 days.

In various embodiments of the above modified oligonucleotides and uses thereof, the modified oligonucleotide comprises or consists of the nucleobase sequence of SEQ ID NO:28. In certain embodiments, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment; and wherein each nucleoside of each wing segment comprises a modified sugar. In particular embodiments, the modified oligonucleotide consists of 20 nucleobases. In certain embodiments, the modified oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; and wherein each cytosine is a 5-methylcytosine. In certain embodiments, the oligonucleotide comprises or consists of the sequence, UCCCGCCTGTGACAUGCAUU (SEQ ID NO:28). In particular embodiments, the oligonucleotide is a full phosphorothioate analog, wherein the oligonucleotide comprises 2′-O-methoxyethyl substitutions at positions 1-6 and 15-20, and wherein residues 7-14 are unmodified 2′-deoxy. In certain embodiments, the oligonucleotide is administered no more than once every 180 days. The modified oligonucleotide may be present in a pharmaceutical composition further comprising one or more pharmaceutically acceptable diluents or excipients.

In various embodiments of the above modified oligonucleotides and uses thereof, an amount of at least 100 μg of the oligonucleotide is administered. In certain embodiments, an amount of between 100 μg and 1500 μg of the oligonucleotide is administered. In certain embodiments, an amount of between 100 μg and 1000 μg of the oligonucleotide is administered. In particular embodiments, the amount of oligonucleotide is administered in a dose of a pharmaceutical composition comprising the oligonucleotide. In particular embodiments of the methods of the present invention, two or more doses are administered, wherein each dose is administered at least 90 days apart.

In various embodiments of the above modified oligonucleotides and uses thereof, the therapeutically effective amount of the oligonucleotide (or composition comprising the oligonucleotide) is administered in two or more doses, wherein each dose is administered at least 90 days apart from the other doses. In various embodiments of the above modified oligonucleotides and uses thereof, a therapeutically effective amount of the oligonucleotide is administered in two or more doses of a pharmaceutical composition comprising the oligonucleotide, wherein each of the two or more doses is administered at least 90 days apart from the other doses. In other related embodiments, the composition is administered in two or more doses, each dose comprising a therapeutically effective amount of the composition, wherein the two or more doses are administered at least 90 days apart from each other.

In various embodiments of the above modified oligonucleotides and uses thereof, the half life of the administered oligonucleotide is at least 44 days. In particular embodiments, the half-life is determined in an ocular tissue selected from retina and choroid.

In various embodiments of the above modified oligonucleotides and uses thereof, the oligonucleotide, or composition comprising the oligonucleotide, is administered by intravitreal injection.

In various embodiments of the above modified oligonucleotides and uses thereof, the duration of action of the oligonucleotide is at least 50 days or at least 100 days.

In various embodiments of the above modified oligonucleotides and uses thereof, the subject is refractory to treatment with one or more of: a steroid, laser treatment, ranibizumab, bevacizumab, and rapamycin.

### BRIEF DESCRIPTION OF THE SEVERAL VIEWS OF THE DRAWINGS

FIG. 1 is a graph depicting the clearance of c-raf antisense oligonucleotides from rabbit retina-choroid following a single intravitreal injection.

FIG. 2 provides optical coherence topography results from a single patient depicting macular thickness (vertical in left panel; horizontal in right panel) at the indicated time points following intravitreal injection of 110 μg of c-raf antisense oligonucleotide. Retinal coherence topography (RCT) measurements (microns) obtained before treatment and 24 weeks post-injection were 528 and 379, respectively. Best corrected visual acuity (VA) determined before treatment and 24 weeks post-injection was 63 letters and 68 letters, respectively.

FIG. 3 provides optical coherence topography results from a single patient depicting macular thickness (vertical in left panel; horizontal in right panel) at the indicated time points following intravitreal injection of 350 μg of c-raf antisense oligonucleotide. Retinal coherence topography (RCT) measurements (microns) obtained before treatment and 24 weeks post-injection were 391 and 228, respectively. Best corrected visual acuity (VA) determined before treatment and 24 weeks post-injection was 49 letters and 55 letters, respectively.

FIG. 4 provides optical coherence topography results from a single patient depicting macular thickness (vertical in left panel; horizontal in right panel) at the indicated time points following intravitreal injection of 700 μg of c-raf antisense oligonucleotide. Retinal coherence topography (RCT) measurements (microns) obtained before treatment and 24 weeks post-injection were 867 and 124, respectively. Best-corrected visual acuity (BCVA) determined before treatment at screening visit and 24 weeks post-injection was 15 letters and 28 letters, respectively.

FIG. 5 provides optical coherence topography results from a single patient depicting macular thickness (vertical in left panel; horizontal in right panel) at the indicated time points following intravitreal injection of 1000 μg of c-raf antisense oligonucleotide. Retinal coherence topography (RCT) measurements (microns) obtained before treatment and 24 weeks post-injection were 423 and 187, respectively. Best-corrected visual acuity (VA) determined before treatment and 24 weeks post-injection was 63 letters and 58 letters, respectively.

FIG. 6 provides a graph summarizing reduction of central retinal thickness (CRT) in patients 24 weeks following initial treatment. The graph shows the percentage of patent having CRT reduction of greater than 600 microns, greater than 200 microns, greater than 100 microns and greater than 0 microns.

FIG. 7 is a graph summarizing reduction of excessive central retinal thickness (CRT) in patients 24 weeks following initial treatment. The graph shows the percentage of patients having reduction of excessive CRT of greater than 90%, greater than 50%, greater than 30% and greater than 0%.

FIG. 8 is a graph summarizing the change in visual acuity in patients 24 weeks following initial treatment. The graph shows the percentage of patients having a greater than 10 letter number change, a greater than 5 letter number change, and stable or improved.

### DETAILED DESCRIPTION

The present invention provides raf antisense oligonucleotides, pharmaceutical compositions, and dosing regimens suitable for using raf antisense oligonucleotides to treat or prevent ocular diseases and disorders, including, e.g., macular edema. In particular embodiments, the raf is c-raf. As demonstrated in the accompanying Examples, c-raf antisense oligonucleotides are effective in reducing retinal thickness in human patients suffering from diabetic macular edema (DME). In addition, the c-raf antisense oligonucleotides are stable following intravitreal administration, which allows them to be administered less frequently, thus reducing the likelihood of injury to the eye caused by intravitreal injection. Furthermore, the c-raf antisense oligonucleotides have been shown to be effective in the treatment of DME refractory to one or more other treatments.

### A. DEFINITIONS

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques can be used for chemical synthesis, and chemical analysis. To the extent permitted, all patents, applications, published applications and other publications, GENBANK Accession Numbers and associated sequence information obtainable through databases such as National Center for Biotechnology Information (NCBI) and other data referred to herein are hereby incorporated by reference in their entirety.

Unless otherwise indicated, the following terms have the following meanings:

“2′-O-methoxyethyl” (also 2′-MOE, 2′-O-(2-methoxyethyl) and 2′-O(CH₂)₂—OCH₃) refers to an O-methoxy-ethyl modification of the 2′ position of a furosyl ring. A 2′-O-methoxyethyl modified sugar is a modified sugar.

“2′-O-methoxyethyl nucleoside” means a nucleoside comprising a 2′-O-methoxyethyl modified sugar moiety.

“3′ target site” refers to the nucleotide of a target nucleic acid which is complementary to the 3′-most nucleotide of a particular antisense compound.

“5′ target site” refers to the nucleotide of a target nucleic acid which is complementary to the 5′-most nucleotide of a particular antisense compound.

“5-methylcytosine” means a cytosine modified with a methyl group attached to the 5′ position. A 5-methylcytosine is a modified nucleobase.

“Administered concomitantly” refers to the co-administration of two agents in any manner in which the pharmacological effects of both are manifest in the patient. Concomitant administration does not require that both agents be administered in a single pharmaceutical composition, in the same dosage form, at the same time or by the same route of administration.

“Administering” means providing a pharmaceutical agent to an individual, and includes, but is not limited to, administering by a medical professional and self-administering.

“Ameliorate” means to make better or improve the symptoms of a condition or disease in a subject.

“Animal” refers to human or non-human animals, including, but not limited to, mice, rats, rabbits, dogs, cats, pigs, horses and non-human primates, including, but not limited to, monkeys and chimpanzees.

“Antisense compound” means an oligomeric compound that is capable of undergoing hybridization to a target nucleic acid through hydrogen bonding.

“Antisense inhibition” means reduction of target nucleic acid or protein levels in the presence of an antisense compound complementary to a target nucleic acid compared to target nucleic acid or protein levels in the absence of the antisense compound.

“Antisense oligonucleotide” means a single-stranded oligonucleotide having a nucleobase sequence that permits hybridization to a complementary region or segment of a target nucleic acid.

“Bicyclic sugar” means a furosyl ring modified by the bridging of two non-geminal ring atoms. A bicyclic sugar is a modified sugar moiety.

“Cap structure” or “terminal cap moiety” means a chemical modification, which has been incorporated at a terminus of an antisense compound. An antisense compound can have both termini “capped”.

“Chimeric antisense compounds” means antisense compounds that have at least 2 chemically distinct regions, each region includes a plurality of subunits.

“Co-administration” means administration of two or more agents to an individual. The two or more agents can be in a single pharmaceutical composition, or can be in separate pharmaceutical compositions. Each of the two or more agents can be administered through the same or different routes of administration. Co-administration encompasses administration in parallel or sequentially.

“Complementarity” means the capacity for pairing between nucleobases of a first nucleic acid and a second nucleic acid.

“Comprise,” “comprises” and “comprising” are to be understood to imply the inclusion of a stated step or element or group of steps or elements but not the exclusion of any other step or element or group of steps or elements.

“Contiguous nucleobases” means nucleobases immediately adjacent to each other.

“Cross-reactive” means an oligomeric compound targeting one nucleic acid sequence can hybridize to a different nucleic acid sequence. For example, in some instances an antisense oligonucleotide targeting human c-raf can cross-react with a murine c-raf. Whether an oligomeric compound cross-reacts with a nucleic acid sequence other than its designated target depends on the degree of complementarity the compound has to the nucleic acid sequence. The higher the complementarity between the oligomeric compound and the non-target nucleic acid, the more likely the oligomeric compound will cross-react with the nucleic acid.

“Cure” means a method that restores health or a prescribed treatment for an illness.

“Deoxyribonucleotide” means a nucleotide having a hydrogen atom at the 2′ position of the sugar portion of the nucleotide. Deoxyribonucleotides can be modified with any of a variety of substituents.

“Designing” or “Designed to” refer to the process of designing an oligomeric compound that specifically hybridizes with a selected nucleic acid molecule or portion thereof.

“Diluent” means an ingredient in a composition that lacks pharmacological activity, but is pharmaceutically necessary or desirable. For example, in drugs that are injected, the diluent can be a liquid, e.g. saline solution.

“Dose” means a specified quantity of a pharmaceutical agent provided in a single administration, or in a specified time period. In certain embodiments, a dose can be administered in two or more boluses, tablets, or injections. For example, in certain embodiments, where ocular administration is desired, the desired dose may require a volume not easily accommodated by a single injection. In such embodiments, two or more injections can be used to achieve the desired dose. In certain embodiments, a dose can be administered in two or more injections to minimize injection site reaction in an individual. Doses can be stated as the amount of pharmaceutical agent per hour, day, week or month.

“Dosage unit” or “unit dosage form” means a form in which a pharmaceutical agent is provided, e.g., pill, tablet, or other dosage unit known in the art. In certain embodiments, a dosage unit is a vial containing lyophilized antisense oligonucleotide. In certain embodiments, a dosage unit is a vial containing reconstituted antisense oligonucleotide.

“Duration” means the period of time during which an activity or event continues. In certain embodiments, the duration of treatment is the period of time during which doses of a pharmaceutical agent are administered.

“Effective amount” or “therapeutically effective amount” in the context of modulating an activity or of treating or preventing a condition means the administration of that amount of active ingredient to a subject in need of such modulation, treatment or prophylaxis, either in a single dose or as part of a series, that is effective for modulation of that effect, or for treatment or prophylaxis or improvement of that condition. The effective amount will vary depending upon the health and physical condition of the subject to be treated, the taxonomic group of subjects to be treated, the formulation of the composition, the assessment of the medical situation, and other relevant factors. “Therapeutically effective amount” or “effective amount” also includes an amount of a pharmaceutical agent that provides a therapeutic benefit to an individual.

“Efficacy” means the ability to produce a desired effect.

“Expression” includes all the functions by which a gene's coded information is converted into structures present and operating in a cell. Such structures include, but are not limited to, the products of transcription and translation.

“First agent” or “first therapeutic agent” means an agent that can be used in combination with a “second agent”. In certain embodiments, the first agent is any antisense compound, oligonucleotide or composition that inhibits c-raf described herein.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid. In certain such embodiments, an antisense oligonucleotide is a first nucleic acid and a target nucleic acid is a second nucleic acid.

“Gapmer” means an antisense compound in which an internal position having a plurality of nucleotides that supports RNaseH cleavage is positioned between external regions having one or more nucleotides that are chemically distinct from the nucleosides of the internal region. A “gap segment” means the plurality of nucleotides that make up the internal region of a gapmer. A “wing segment” means the external region of a gapmer.

“Gap-widened” means an antisense compound has a gap segment of 12 or more contiguous 2′-deoxyribonucleotides positioned between and immediately adjacent to 5′ and 3′ wing segments of from one to six nucleotides having modified sugar moieties.

“Hybridization” means the annealing of complementary nucleic acid molecules. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

“Immediately adjacent” means there are no intervening nucleotides between the immediately adjacent elements. For example, between regions, segments, nucleotides and/or nucleosides.

“Induce”, “inhibit”, “potentiate”, “elevate”, “increase”, “decrease” or the like, e.g., denote quantitative differences between two states. For example, “an amount effective to inhibit the activity or expression of c-raf” means that the level of activity or expression of c-raf in a treated sample will differ from the level of c-raf activity or expression in untreated cells. Such terms are applied to, for example, levels of expression, and levels of activity.

“Inhibiting the expression or activity” refers to a reduction, blockade of the expression or activity of the target and does not necessarily indicate a total elimination of expression or activity.

“Internucleoside linkage” refers to the chemical bond between nucleosides.

“Linked nucleosides” means adjacent nucleosides which are bonded together. “Mismatch” refers to a non-complementary nucleobase within a complementary oligomeric compound.

“Modified internucleoside linkage” refers to a substitution and/or any change from a naturally occurring internucleoside bond (i.e. a phosphodiester internucleoside bond).

“Modified nucleobase” means any nucleobase other than adenine, cytosine, guanine, thymidine, or uracil. An “unmodified nucleobase” means the purine bases, adenine (A) and guanine (G), and the pyrimidine bases, thymine (T), cytosine (C) and uracil (U).

“Modified oligonucleotide” means an oligonucleotide comprising a modified internucleoside linkage, a modified sugar, and/or a modified nucleobase. A modified oligonucleotide can also have a nucleoside mimetic or nucleotide mimetic.

“Modified sugar” refers to a substitution and/or any change from a natural sugar.

“Modulation” means a perturbation of function, for example, one associated with either an increase (stimulation or induction) or a decrease (inhibition or reduction) in expression.

“Monomer” refers to a single unit of an oligomer. Monomers include, but are not limited to, nucleosides and nucleotides, whether naturally occurring or modified.

“Motif” means the pattern of unmodified and modified nucleosides in an antisense compound.

“Naturally occurring internucleoside linkage” means a 3′ to 5′ phosphodiester linkage.

“Natural sugar” means a sugar found in DNA (2′-H) or RNA (2′-OH).

“Nucleic acid” refers to molecules composed of monomeric nucleotides. A nucleic acid includes, but is not limited to, ribonucleic acids (RNA), deoxyribonucleic acids (DNA), single-stranded nucleic acids, double-stranded nucleic acids, small interfering ribonucleic acids (sRNA), and microRNAs (miRNA).

“Nucleobase” means a heterocyclic moiety capable of pairing with a base of another nucleic acid.

“Nucleobase complementarity” refers to a nucleobase that is capable of base pairing with another nucleobase. For example, in DNA, adenine (A) is complementary to thymine (T). For example, in RNA, adenine (A) is complementary to uracil (U). In certain embodiments, complementary nucleobase refers to a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair.

“Nucleobase sequence” means the order of contiguous nucleobases independent of any sugar, linkage, and/or nucleobase modification.

“Nucleoside” means a nucleobase linked to a sugar.

“Nucleotide” means a nucleoside having a phosphate group covalently linked to the sugar portion of the nucleoside.

“Nucleoside mimetic” includes those structures used to replace the sugar or the sugar and the base and not necessarily the linkage at one or more positions of an oligomeric compound such as, for example, nucleoside mimetics having morpholino, cyclohexenyl, cyclohexyl, bicyclo or tricyclo sugar mimetics, e.g. non furanose sugar units.

“Nucleotide mimetic” includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage).

“Oligomeric compound” means a polymer of linked monomeric subunits which is capable of hybridizing to at least a region of a nucleic acid molecule.

“Oligonucleotide” means an oligomer or polymer of linked nucleoside or nucleotide monomers each of which can be modified or unmodified, independent one from another. The term “oligonucleotide” includes oligomers comprising non-naturally occurring monomers, or portions thereof. Such modified or substituted oligonucleotides are often preferred over native forms because of properties such as, for example, enhanced cellular uptake and increased stability in the presence of nucleases.

“Parenteral administration,” means administration by a manner other than through the digestive tract, e.g., through topical administration, injection or infusion. Parenteral administration includes, but is not limited to, subcutaneous administration, intravenous administration, and intramuscular administration.

“Pharmaceutically acceptable carrier” or “Pharmaceutically acceptable diluent” means a carrier or diluent that does not interfere with the structure of the oligonucleotide. Certain of such carries enable pharmaceutical compositions to be formulated as, for example, tablets, pills, dragees, capsules, liquids, gels, syrups, slurries, suspension and lozenges for the oral ingestion by a subject. For example, a pharmaceutically acceptable carrier can be a sterile aqueous solution.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Pharmaceutical composition” or “composition” means a mixture of substances suitable for administering to an animal. For example, a composition can comprise one or more antisense oligonucleotides and a sterile aqueous solution.

“Phosphorothioate internucleoside linkage” or “phosphorothioate linkage” means a linkage between nucleosides where the phosphodiester bond is modified by replacing one of the non-bridging oxygen atoms with a sulfur atom. A phosphorothioate linkage is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent,” “prevention” or “preventing” refers to inhibiting, delaying or forestalling the onset or development of a condition or disease for a period of time from hours to days, preferably weeks to months to years or permanently. It can also mean reducing the likelihood that a condition or disease will occur during a period of time.

“Prodrug” means a therapeutic agent that is prepared in an inactive form that is converted to an active form (i.e., drug) within the body or cells thereof by the action of endogenous or non-endogenous enzymes or other chemicals and/or conditions.

“Region” is defined as a portion of the target nucleic acid having at least one identifiable structure, function, or characteristic.

“Ribonucleotide” means a nucleotide having a hydroxy at the 2′ position of the sugar portion of the nucleotide. Ribonucleotides can be modified with any of a variety of substituents.

“Salts” mean physiologically and pharmaceutically acceptable salts of antisense compounds, i.e., salts that retain the desired biological activity of the parent oligonucleotide and do not impart undesired toxicological effects thereto.

“Second agent” or “second therapeutic agent” means an agent that can be used in combination with a “first agent”. In certain embodiments, a second therapeutic agent can be any agent that inhibits or prevents c-raf or VEGF expression or activity. A second therapeutic agent can include, but is not limited to, an siRNA or antisense oligonucleotide.

“Segments” are defined as smaller, sub-portions of regions within a target nucleic acid. “Shortened” or “truncated” versions of antisense oligonucleotides or target nucleic acids taught herein have one, two or more nucleosides deleted.

“Side effects” mean physiological responses attributable to a treatment other than desired effects. In certain embodiments, side effects include, without limitation, injection site reactions, liver function test abnormalities, renal function abnormalities, liver toxicity, renal toxicity, central nervous system abnormalities, and myopathies. For example, increased aminotransferase levels in serum can indicate liver toxicity or liver function abnormality. For example, increased bilirubin can indicate liver toxicity or liver function abnormality.

“Single-stranded oligonucleotide” means an oligonucleotide which is not hybridized to a complementary strand. “Single-stranded modified oligonucleotide” means a modified oligonucleotide which is not hybridized to a complementary strand.

“siRNA” is defined as a double-stranded compound having a first and second strand and comprises a central complementary portion between said first and second strands and terminal portions that are optionally complementary between said first and second strands or with a target mRNA. In one non-limiting example, the first strand of the siRNA is antisense to the target nucleic acid, while the second strand is complementary to the first strand. Once the antisense strand is designed to target a particular nucleic acid target, the sense strand of the siRNA can then be designed and synthesized as the complement of the antisense strand and either strand can contain modifications or additions to either terminus.

“Sites,” as used herein, are defined as unique nucleobase positions within a target nucleic acid.

“Slows progression” means a decrease in the development of a disease, condition or symptom.

“Specifically hybridizable” means an antisense compound that hybridizes to a target nucleic acid to induce a desired effect, while exhibiting minimal or no effects on non-target nucleic acids.

“Subject” means a human or non-human animal selected for treatment or therapy.

“Targeted” or “targeted to” means having a nucleobase sequence that will allow specific hybridization of an antisense compound to a target nucleic acid to induce a desired effect.

“Target nucleic acid,” “target RNA,” “target RNA transcript” and “nucleic acid target” all mean a nucleic acid capable of being targeted by antisense compounds.

“Targeting” means the process of design and selection of an antisense compound that will specifically hybridize to a target nucleic acid and induce a desired effect.

“c-raf nucleic acid” means any nucleic acid encoding c-Raf. For example, in certain embodiments, a c-raf nucleic acid includes, without limitation, a DNA sequence encoding c-Raf, an RNA sequence transcribed from DNA encoding c-Raf, and an mRNA sequence encoding c-Raf. “c-raf mRNA” means an mRNA encoding a c-Raf protein.

“Treatment” refers to administering a composition of the invention to effect an alteration or improvement of a disease, condition or symptom.

“Unmodified nucleotide” means a nucleotide composed of naturally occurring nucleobases, sugar moieties and internucleoside linkages. In certain embodiments, an unmodified nucleotide is an RNA nucleotide (i.e., β-D-ribonucleosides) or a DNA nucleotide (i.e., β-D-deoxyribonucleoside).

“Wing segment” means a plurality of nucleosides modified to impart to an oligonucleotide properties such as enhanced inhibitory activity, increased binding affinity for a target nucleic acid, or resistance to degradation by in vivo nucleases.

### B. METHODS OF TREATING AND PREVENTING OCULAR DISEASES AND DISORDERS USING RAF ANTISENSE OLIGONUCLEOTIDES

Methods of the present invention may be used to treat, prevent, or inhibit an ocular disease or disorder in a subject diagnosed with or at risk for developing an ocular disease or disorder. Similarly, they may be used to reduce the incidence of or likelihood of occurrence of an ocular disease or disorder in a subject at risk for developing an ocular disease or disorder. In certain embodiments, the subject is diagnosed with DME or diabetes and is, therefore, considered to be at risk for developing DME. The subject is preferably a mammal, such as a human. Accordingly, in particular embodiments of methods of the present invention related to inhibiting an ocular disease or disorder, a subject is first determined to be at risk of developing an ocular disease or disorder. For example, a patient may be determined to have diabetes.

In particular embodiments, methods of the present invention are used to treat patients who are refractory to one or more other treatments for the ocular disease or disorder afflicting the subject. For example, the patient may not have responded favorably to another treatment, or a patient may have initially responded favorably to another treatment but is no longer responding favorably to the treatment. The previous treatment may be any treatment suitable for the ocular disease or disorder from which the patient is suffering. In particular embodiments, the patient was diagnosed with a macular edema, e.g., diabetic macular edema, and did not respond favorably to or is no longer responding favorably to treatment with one or more of pegaptanib, ranibizumab, bevacizumab, rapamycin, fluocinolone acetonide, corticosteroids, siRNAs (e.g., siRNA-027 or bevasiranib (Cand5)), RTP801i, aflibercept (VEGF Trap; Regeneron, Tarrytown, N.Y.), a locked nucleic acid-based HIF-1 alpha inhibitor developed by Santaris Pharma NS (Denmark; e.g., EZN2968-HIF-1alpha), or laser, e.g., laser photocoagulation. Pegaptanib sodium (Macugen®, Eyetech Pharmaceuticals, Melville, N.Y./Pfizer, New York, N.Y.) is an anti-VEGF aptamer, a small piece of RNA that self-folds into a shape that binds to and blocks the effects of VEGF₁₆₅, one isoform of the VEGF family of molecules. Ranibizumab (Lucentis™, Genentech, San Francisco, Calif.) is an antibody fragment that also binds and blocks the effects of VEGF. Unlike pegaptanib, ranibizumab binds and inhibits all isoforms of VEGF. Bevacizumab (Avastin®, Genentech, San Francisco, Calif.) is the full antibody from which ranibizumab is derived. Accordingly, in particular embodiments, methods of the present invention include the step of determining that a subject is refractory to one or more treatments for an ocular disease or disorder. In particular embodiments, the patient is refractory to treatment with a therapeutic agent that inhibits vascular endothelial growth factor (VEGF). In particular embodiment, the patient is diagnosed with macular edema and is refractory to treatment with a therapeutic agent that inhibits vascular endothelial growth factor (VEGF).

An “ocular disorder” herein is a disease or disorder involving the eye. Ocular disorders include, but are not limited to, various proliferative diseases like proliferative vitreoretinopathy, infectious diseases as endophthalmitis, viral infections, e.g., CMV retinitis, herpes simplex, varicella-zoster, Epstein-Barr, and adenovirus, inflammatory diseases such as uveitis or intraocular inflammation, vascular diseases such as diabetic retinopathy, diabetic macular edema, cystoid macular edema, corneal neovascularization, age-related macular degeneration, retinal vein or arterial occlusion, e.g., branch retinal vein occlusion or central retinal vein occlusion, viral infections, e.g., CMV retinitis, herpes simplex, varicella-zoster, Epstein-Barr, and adenovirus, and degenerative disorders such as age-related macular degeneration and retinitis pigmentosa. In addition, diseases like glaucoma or optic neuritis might be potentially treated with the effective delivery of neuroprotective agents to the retina and optic nerve. In particular embodiments, the ocular disease or disorder is diabetic macular edema, cystoid macular edema, age related macular degeneration, diabetic retinopathy, or corneal neovascularization. In one embodiment, the ocular disease or disorder is neovascular glaucoma.

In particular embodiments, the disease or disorder is a macular edema, e.g., diabetic macular edema (DME) or cystoid macular edema. Macular edema occurs when fluid and protein deposits collect on or under the macula of the eye, causing it to thicken and swell. The macula is located near the center of the retina at the back of the eyeball, which is an area that holds tightly packed cones that provide clear central vision to see form, color, and detail directly in the line of sight. Macular edema distorts this central vision.

Retinal blood vessel obstruction, eye inflammation, and age-related macular degeneration have all been associated with macular edema. The macula may also be affected by swelling following cataract extraction, though typically this resolves itself naturally. Macular edema is common in diabetics, and the lifetime risk for diabetics to develop macular edema is about 10%. The condition is closely associated with the degree of diabetic retinopathy (retinal disease). Hypertension and fluid retention also increase the hydrostatic pressure within capillaries which drives fluid from within the vessels into the retina. A common cause of fluid retention in diabetes is kidney disease with loss of protein in the urine (proteinuria).

Diabetic macular edema (DME) is classified into focal and diffuse types. Focal macular edema is caused by foci of vascular abnormalities, primarily microaneurysms, which tend to leak fluid whereas diffuse macular edema is caused by dilated retinal capillaries in the retina. Two types of laser treatment for diabetic macular edema are focal and grid. Focal laser treatment is used to treat focal diabetic macular edema; the aim is to close leaking microaneurysms. Grid laser treatment is used to treat diffuse diabetic macular edema and is applied to areas of retinal thickening in which there is diffuse leakage; the aim is to produce a retinal burn of mild to moderate intensity.

Cystoid macular edema (CME) affects the central retina or macula. When this condition is present, multiple cyst-like (cystoid) areas of fluid appear in the macula and cause retinal swelling or edema. CME may accompany a variety of diseases such as retinal vein occlusion, uveitis, or diabetes. It most commonly occurs after cataract surgery. About 1-3% of patients who have cataract extractions suffer decreased vision due to CME during the first post-operative year, usually from two to four months after cataract surgery.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the raf antisense oligonucleotides of the present invention, e.g., c-raf antisense oligonucleotides, are especially useful in reducing the severity of AMD. The progress of this therapy is easily monitored by conventional techniques and assays, including, for example, angiography or optical coherence tomography (OCT).

Macular degeneration affects between five and ten million patients in the United States, and it is the leading cause of blindness worldwide. Macular degeneration affects central vision and causes the loss of photoreceptor cells in the central part of retina called the macula. Macular degeneration can be classified into two types: dry type and wet type. The dry form is more common than the wet, with about 90% of age-related macular degeneration (ARMD) patients diagnosed with the dry form. The wet form of the disease and geographic atrophy, which is the end-stage phenotype of dry ARMD, lead to more serious vision loss. All patients who develop wet form ARMD previously had dry form ARMD for a prolonged period of time. The exact causes of age-related macular degeneration are still unknown. The dry form of ARMD may result from the aging and thinning of macular tissues and from deposition of pigment in the macula. In wet ARMD, new blood vessels grow beneath the retina and leak blood and fluid. This leakage causes the retinal cells to die, creating blind spots in central vision.

For the vast majority of patients who have the dry form of macular degeneration, no treatment is available. Because the dry form precedes development of the wet form of macular degeneration, intervention in disease progression of the dry form could benefit patients that presently have dry form and may delay or prevent development of the wet form.

Diabetic retinopathy occurs when diabetes damages blood vessels inside the retina. Non-proliferative retinopathy is a common, usually mild form that generally does not interfere with vision. Abnormalities are limited to the retina, and vision is impaired only if the macula is involved. If left untreated, it can progress to proliferative retinopathy, the more serious form of diabetic retinopathy. Proliferative retinopathy occurs when new blood vessels proliferate in and around the retina. Consequently, bleeding into the vitreous, swelling of the retina, and/or retinal detachment may occur, leading to blindness.

Methods and compositions of the present invention may be used to treat an existing ocular disease or disorder or to prevent or inhibit the development of an ocular disease or disorder. In one embodiment, the present invention includes a method of treating, preventing, inhibiting, or reducing the incidence or likelihood of occurrence of an ocular disease or disorder in a subject comprising administering two or more doses of a pharmaceutical composition comprising a raf antisense oligonucleotide, e.g., a c-raf antisense oligonucleotide, to a subject's eye, wherein the doses are administered at least 90 days apart from each other. In particular embodiments, the doses are administered at least 180 days apart from each other. In related embodiments, the doses are administered at least 120 days, at least 150 days, at least 210 days, at least 240 days, at least 270 days, at least 300 days, at least 330 days, or at least 360 days apart from each other. In one embodiment, the doses are administered at least one year apart from each other. In related embodiments, the doses are administered at least 3 months, at least 4 months, at least 5 months, at least 6 months, at least 7 months, at least 8 months, at least 9 months, at least 10 months, at least 11 months, or at least 12 months apart from each other. In further related embodiments, the doses are administered between 3 months and 12 months apart, between 3 months and 9 months apart, between 3 months and 6 months apart, between 4 months and 12 months apart, between 4 months and 9 months apart, between 4 months and 6 months apart, between 6 months and 12 months apart, between 6 months and 9 months apart, or between 9 months and 12 months apart. One or both of a subject's eyes may be treated. As described herein, the two or more doses are administered to the same eye, such that if both eyes were being treated, then the method would be practiced on each eye.

In particular embodiments, the amount of raf antisense oligonucleotide, e.g., c-raf antisense oligonucleotide, administered in each dose is between 100 μg and 1000 μg, between 200 μg and 1000 μg, between 300 μg and 1000 μg, between 500 μg and 1000 μg, between 100 μg and 1500 μg, between 200 μg and 1500 μg, between 300 μg and 1500 μg, between 500 μg and 1500 μg between 100 μg and 2000 μg, between 200 μg and 2000 μg, between 300 μg and 2000 μg, between 500 μg and 2000 μg, equal or greater than 100 μg, equal or greater than 200 μg, equal or greater than 300 μg, equal or greater than 400 μg, equal or greater than 500 μg, or equal or greater than 1000 μg per eye. In certain embodiments, the amount of raf antisense oligonucleotide, e.g., c-raf antisense oligonucleotide, administered in each dose is about 110 μg, about 350 μg, about 700 μg, about 1000 μg, or about 1400 μg.

In particular embodiments, the doses are administered by intravitreal injection. In particular embodiments, the intravitreal concentration of the raf antisense oligonucleotide, e.g., c-raf antisense oligonucleotide, following intravitreal administration is between 3 μM and 30 μM, between 6 μM and 30 μM, between 9 μM and 30 μM, between 15 μM and 30 μM, equal or greater than 3 μM, equal or greater than 6 μM, equal or greater than 9 μM, equal or greater than 12 μM, or equal or greater than 15 μM. In one embodiment, the intravitreal concentration of raf antisense oligonucleotide, e.g., c-raf antisense oligonucleotide, is about 10 μM.

In particular embodiments, the ocular disease or disorder is a macular edema, such as diabetic macular edema or cystoid macular edema.

In certain embodiments, methods of the present invention utilize a modified c-raf antisense oligonucleotide comprising or consisting of a sequence 100% complementary to nucleobases 2771 to 2790 of SEQ ID NO:29, wherein the composition is administered no more than once every 90 days. In one embodiment, the modified oligonucleotide comprises or consists of the nucleobase sequence of SEQ ID NO:28. In certain embodiments, the modified oligonucleotide comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment; and wherein each nucleoside of each wing segment comprises a modified sugar. In particular embodiments, the modified oligonucleotide consists of 20 nucleobases. In certain embodiments, the modified oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; and wherein each cytosine is a 5-methylcytosine.

In certain embodiments, the c-raf antisense oligonucleotide comprises or consists of the sequence, UCCCGCCTGTGACAUGCAUU (SEQ ID NO:28). In particular embodiments, the oligonucleotide is a full phosphorothioate analog. In additional embodiments, this oligonucleotide comprises one or more 2′-O-methoxyethyl substitutions. In one embodiment, the oligonucleotide comprises or consists of the sequence, UCCCGCCTGTGACAUGCAUU (SEQ ID NO:28), wherein the oligonucleotide is a full phosphorothioate analog, wherein the oligonucleotide comprises 2′-O-methoxyethyl substitutions at positions 1-6 and 15-20, and wherein residues 7-14 are unmodified 2′-deoxy.

In one particular embodiment, the present invention includes a method of treating macular edema in a subject, comprising administering two or more doses of a pharmaceutical composition comprising a modified c-raf antisense oligonucleotide 100% complementary to nucleobases 2771 to 2790 of SEQ ID NO:29, wherein said modified c-raf oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; wherein each cytosine is a 5-methylcytosine, to the eye of the subject by intravitreal injection, wherein each dose consists of between 100 μg and 1000 μg or between 200 μg and 1000 μg of the oligonucleotide, and wherein the two or more doses are administered at least 90 days apart from each other. In one embodiment, each dose consists of about 350 μg of the oligonucleotide. Other doses described herein may also be used to practice this and any other methods described herein. In particular embodiments, the two doses are administered at least 120 or 180 days apart from each other, between 90 days and 120 days apart from each other, between 90 days and 150 days apart from each other, between 90 days and 180 days apart from each other, between 90 days and 365 days apart from each other, or between 180 days and 365 days apart from each other. In related embodiments, two or more doses are administered about once every two months, about once every three months, about once every four months, about once every five months, or about once every six months. In certain embodiments, doses are administered at a frequency of one of the following ranges: every two to three months, every two to four months, every two to five months, every two to six months, every three to four months, every three to five months, every three to six months, every four to five months, or every five to six months.

In one embodiment, the patient is refractory to treatment with one or more other therapies or drugs used to treat macular edema, e.g., laser, steroids, ranibizumab, bevacizumab, or rapamycin. Thus, particular embodiments include the step of identifying a patient as being refractory to one of these other treatments before administering to the patient the c-raf antisense oligonucleotide.

In a related embodiment, the method described above is practiced using a c-raf antisense oligonucleotide comprising or consisting of the sequence, UCCCGCCTGTGACAUGCAUU (SEQ ID NO:28). In particular embodiments, the oligonucleotide is a full phosphorothioate analog. In additional embodiments, this oligonucleotide comprises one or more 2′-O-methoxyethyl substitutions. In one embodiment, the oligonucleotide comprises or consists of the sequence, UCCCGCCTGTGACAUGCAUU (SEQ ID NO:28), wherein the oligonucleotide is a full phosphorothioate analog, wherein the oligonucleotide comprises 2′-O-methoxyethyl substitutions at positions 1-6 and 15-20, and wherein residues 7-14 are unmodified 2′-deoxy.

Any of the methods described above may also be practiced to reduce retinal thickness, reduce excess retinal thickness, and/or improve visual acuity. In particular embodiments, retinal thickness is reduced by at least 25, at least 50, at least 100, or at least 150 microns. In various embodiments, retinal thickness is determined as vertical thickness or as horizontal thickness. Excess retinal thickness is defined as retinal thickness exceeding 212 microns.

In particular embodiments, visual acuity is increased by at least 10%, at least 25%, at least 50%, at least 75%, or at least 100%. In particular embodiments, visual acuity is determined according to the Early Treatment Diabetic Retinopathy Study (ETDRS), a highly accurate criteria for visual acuity determination based upon exact light intensity, distance etc., which is used in most clinical studies. This test gives a total number of letters seen by a patient. The ETDRS test, based on the Bailey-Lovie layout, implemented with Sloan letters, was developed to establish a standardized method of visual acuity measurement for the Early Treatment of Diabetic Retinopathy Study (ETDRS). ETDRS charts were used in all subsequent clinical studies, and did much to familiarize the profession with the new layout and progression. Data from the ETDRS were used to select letter combinations that give each line the same average difficulty, without using all letters on each line. In particular embodiments, the total number of additional letters seen by a patient is increased by at least 2, at least 3, at least 4, at least 5, at least 6, at least 7, at least 8, at least 9, or at least 10 letters upon treatment according to a method of the present invention. In particular embodiments, visual acuity is considered increased when the total number of additional letters seen by a patient is increased by at least 2, at least 3, at least 4, at least 5, at least 6, at least 7, at least 8, at least 9, or at least 10 letters.

In certain embodiments, visual acuity may be measured using the Snellen visual acuity test, which compares the visual acuity of a test patient to a normal control when reading from 20 feet. In the Snellen fraction 20/20, the first number represents the test distance, 20 feet. The second number represents the distance that the average eye can see the letters on a certain line of the eye chart. So, 20/20 means that the eye being tested can read a certain size letter when it is 20 feet away. If a person sees 20/40, at 20 feet from the chart that person can read letters that a person with 20/20 vision could read from 40 feet away. The 20/40 letters are twice the size of 20/20 letters; however, it does not mean 50% vision since 20/20 sounds like it is one half of 20/40. If 20/20 is considered 100% visual efficient, 20/40 visual acuity is 85% efficient. Best-corrected visual acuity is the visual acuity of a person wearing the best glasses or contact lens prescription for that person.

Raf antisense oligonucleotides, e.g., c-raf antisense oligonucleotides, of the present invention may be used according to any of the methods described above alone or in combination with one or more additional therapies, e.g., laser photocoagulation, vitrectomy surgery, or treatment with another therapeutic agent. Raf antisense oligonucleotides, e.g., c-raf antisense oligonucleotides, of the present invention of the present invention may be administered in combination with a second therapeutic agent for a variety of reasons, including increased efficacy or to reduce undesirable side effects. In particular embodiments, the raf antisense oligonucleotide and the one or more additional therapeutic agents act synergistically to treat or inhibit an ocular disease or disorder, such as macular edema. The raf antisense oligonucleotide may be administered prior to, subsequent to, or simultaneously with the additional therapeutic agent. The raf antisense oligonucleotide may be delivered in a separate formulation or in the same formulation as the additional raf antisense oligonucleotide.

In certain embodiments, methods of the present invention comprise administering a c-raf antisense oligonucleotide in combination with one or more additional ocular drugs. Ocular drugs include a variety of different types of molecules, including but not limited to peptides, polypeptides, polynucleotides, antibodies, small organic compounds, metals, small inorganic molecules and radionuclides.

In particular embodiments, the ocular drug co-administered with a raf antisense oligonucleotide of the present invention is an antiangiogenic factor, such as an inhibitor of vascular endothelial growth factor, e.g., pegaptanib sodium injection (Macugen™), bevacizumab (Avastin™), Sirna-027™, bevasiranib (Cand5™), aflibercept (VEGF-TRAP), Envison™, ranibizumab (Lucentis™), or another formulation of the active ingredient of any of these drugs. Aflibercept (VEGF Trap; Regeneron, Tarrytown, N.Y.) is an anti-angiogenic fusion protein specifically designed to bind all forms of vascular endothelial growth factor-A (VEGF-A). A variety of different types of agents directed against VEGF may be used in combination therapies according to the invention, including, e.g., antisense, siRNAs, ribozymes, antibodies, shRNAs, VEGF tyrosine kinase inhibitors, Src kinase inhibitors and photodynamic therapy such as verteporfin (Visudyne™), Photofrin™ and texaphyrins.

Other ocular drugs useful in combination with a raf antisense oligonucleotide according to methods of the present invention include, e.g., anecortave acetate suspension (Retaane™), Envizon™, Combretastatin™, AdPEDF, kringle5, ganciclovir, ketotifen, verteporfin, pegaptanib, anecortave acetate, dexamethasone, rapamycin, fluocinolone acetonide, triamcinolone, and lerdelimumab, Macugen™, Lucentis™, and Avastin™. Additional ocular drugs useful in combination with a raf antisense oligonucleotide according to methods of the present invention include, e.g., a locked nucleic acid-based HIF-1 alpha inhibitor developed by Santaris Pharma NS (Denmark; e.g., EZN2968-HIF-1alpha). Other representative ocular drugs that may be used according to the present invention include, but are not limited to, corticosteroids, RTP801i, siRNA-027 and those described in Bartlett, J. D and Jaanus, S., General Ophthalmology, Clinical Ocular Pharmacology, (Butterwoth-Heinemann, 1995) and Hom, M. M., Mosby's Ocular Drug Consult.

In certain embodiments, the ocular drug coadministered with a raf antisense oligonucleotide downregulates the expression or activity of one or more growth factors, e.g., vascular endothelial growth factor (VEGF), erythropoietin (EPO), hepatocyte growth factor (HGF), an angiopoietin (e.g., angiopoietin-2), and/or basic fibroblast growth factor (bFGF).

In particular embodiments, a raf antisense oligonucleotide is administered to a subject having a macular edema, e.g., diabetic macular edema, in combination with pegaptanib, ranibizumab, rapamycin, fluocinolone acetonide, steroid, or laser, e.g., laser photocoagulation.

In particular embodiments, bevacizumab, ranibizumab, or rapamycin are administered at a dosage of between 0.1 mg and 2.0 mg about every thirty days. For example, in particular embodiments, bevacizumab may be administered by monthly intravitreal injection of 1.25 mg, and ranibizumab may be administered by monthly intravitreal injection of 0.3 or 0.5 mg.

In particular embodiments, raf antisense oligonucleotides and any additional therapeutic agent are administered directly to the eye. The human eye can be divided into the anterior and posterior anatomical segments. Drug delivery to the anterior segment is primarily achieved through topical application, and significant success has been achieved in delivering drugs to this area. However, the delivery of drugs to the posterior segment of the eye poses a great challenge. Currently, the posterior segment disease treatment focuses on four approaches to deliver drugs—topical, systemic, intraocular, and periocular, including subconjunctival, subtenon, and retrobulbar modes of administration. Any of these routes of administered may be used according to the methods of the present invention, including the particular routes of administration described in further detail below.

Topical application of drugs for treatment of posterior eye disorders is not very effective due to the long diffusional path length, rapid precorneal elimination due to solution drainage, normal or induced lacrimation, and corneal epithelial impermeability to molecules larger than 5 kDa. Although the systemic approach can deliver drugs to the eye, systemically administered drugs have poor access to the eye tissues because of the blood-aqueous barrier (which prevents the substances from entering into the aqueous humor) and because of the blood-retinal barrier (which severely limits drug entry into the extravascular space of the retina and into the vitreous). Consequently, large systemic doses are required, and this can induce toxicity and unwanted side effects. Intravitreal injections are an effective way of delivering drugs to the vitreoretinal region. However, intravitreal injections can potentially induce retinal detachment, hemorrhage, endophthalmitis, and cataracts, particularly when frequent drug administration is required. Accordingly, the methods of the present invention, which provide for drug administration no more than once every 90 days, are well-suited to intravitreal administration.

Periocular modes of administration include subconjunctival, subtenon, and retrobulbar. In all of these modes, the drug is interfaced with sclera. There is substantial evidence indicating that drugs administered subconjunctivally can reach the vitreous effectively. The sclera does not provide an effective barrier to the entry of drugs, and even solutes of relatively large molecular weight can penetrate through it. The drugs can gain entry into the posterior segments from the subconjunctival space after entering the sclera. Systemic absorption is low with subconjunctival route, which can lower systemic side effects while providing a localized drug effect.

### C. RAF ANTISENSE OLIGONUCLEOTIDES

The present invention employs oligonucleotides targeted to nucleic acids encoding a raf family member, which modulate raf gene expression. In particular embodiments, these raf antisense oligonucleotides reduce or inhibit the expression of the targeted raf family member. In various embodiments, the oligonucleotides target c-raf (raf-1); however, compositions and methods for modulating expression of other forms of raf also have utility and are comprehended by this invention. In particular embodiments, the raf antisense oligonucleotide targets human c-raf. Exemplary raf antisense oligonucleotides that may be used according to the present invention are described herein and also in U.S. Pat. Nos. 5,563,255, 5,952,229, 6,358,932, 5,656,612, 5,919,773, 6,410,518, and 6,806,258.

The relationship between an oligonucleotide and its complementary nucleic acid target to which it hybridizes is commonly referred to as “antisense.” “Targeting” an oligonucleotide to a chosen nucleic acid target, in the context of this invention, is a multistep process. The process usually begins with identifying a nucleic acid sequence whose function is to be modulated. This may be, as examples, a cellular gene (or mRNA made from the gene) whose expression is associated with a particular disease state, or a foreign nucleic acid from an infectious agent. In the present invention, the target is a nucleic acid encoding raf; in other words, the raf gene or mRNA expressed from the raf gene. The targeting process also includes determination of a site or sites within the nucleic acid sequence for the oligonucleotide interaction to occur such that the desired effect—modulation of gene expression—will result. Once the target site or sites have been identified, oligonucleotides are chosen which are sufficiently complementary to the target, i.e., hybridize sufficiently well and with sufficient specificity, to give the desired modulation.

Raf antisense oligonucleotides inhibit or reduce expression of the raf mRNA and/or protein. Inhibition of raf expression can be measured in ways which are routine in the art, for example by Northern blot assay of mRNA expression or Western blot assay of protein expression as taught in the examples of the instant application. Effects on cell proliferation or retinal thickness can also be measured, as taught in the examples of the instant application. “Hybridization,” in the context of this invention, means hydrogen bonding, also known as Watson-Crick base pairing, between complementary bases, usually on opposite nucleic acid strands or two regions of a nucleic acid strand. Guanine and cytosine are examples of complementary bases which are known to form three hydrogen bonds between them. Adenine and thymine are examples of complementary bases which form two hydrogen bonds between them. “Specifically hybridizable” and “complementary” are terms which are used to indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable. An oligonucleotide is specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment, or, in the case of in vitro assays, under conditions in which the assays are conducted.

In certain embodiments of this invention, oligonucleotides are provided which are targeted to mRNA encoding c-raf, B-raf or A-raf. In accordance with this invention, persons of ordinary skill in the art will understand that mRNA includes not only the coding region which carries the information to encode a protein using the three letter genetic code, but also associated ribonucleotides which form a region known to such persons as the 5′-untranslated region, the 3′-untranslated region, the 5′ cap region, intron regions and intron/exon or splice junction ribonucleotides. Thus, oligonucleotides may be formulated in accordance with this invention that are targeted wholly or in part to these associated ribonucleotides as well as to the coding ribonucleotides. In certain embodiments, the oligonucleotide is targeted to a translation initiation site (AUG codon) or sequences in the 5′- or 3′-untranslated region of the human c-raf mRNA. The functions of messenger RNA to be interfered with include all vital functions such as translocation of the RNA to the site for protein translation, actual translation of protein from the RNA, splicing or maturation of the RNA and possibly even independent catalytic activity which may be engaged in by the RNA. The overall effect of such interference with the RNA function is to cause interference with raf protein expression.

In particular embodiments, an oligonucleotide of the present invention targets a human c-raf polynucleotide or mRNA, e.g., the oligonucleotide is fully complementary to a region of a human c-raf mRNA. The sequence of an exemplary human c-raf mRNA is provided in SEQ ID NO:29, which corresponds to the sequence provided at GenBank Accession No. NM_{—}002880.3. In other embodiments, an oligonucleotide of the present invention targets a region of a human A-raf mRNA. The sequence of an exemplary human A-raf mRNA is provided in SEQ ID NO:30, which corresponds to the sequence provided at GenBank Accession No. NM_{—}001654.3. In other embodiments, an oligonucleotide of the present invention targets a region of a human B-raf mRNA. The sequence of an exemplary human B-raf mRNA is provided in SEQ ID NO:31, which corresponds to the sequence provided at GenBank Accession No. NM_{—}004333.

In particular embodiments, an oligonucleotide of the present invention comprises one or more modifications. It is not necessary for all positions in a given compound to be uniformly modified, and in fact more than one of the modifications described infra may be incorporated in a single compound or even at a single nucleoside within an oligonucleotide. Certain oligonucleotides of this invention are chimeric oligonucleotides. “Chimeric oligonucleotides” or “chimeras”, in the context of this invention, are oligonucleotides which contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the RNA target) and a region that is a substrate for RNase H cleavage. In one embodiment, a chimeric oligonucleotide comprises at least one region modified to increase target binding affinity, and, usually, a region that acts as a substrate for RNAse H. Affinity of an oligonucleotide for its target (in this case a nucleic acid encoding raf) is routinely determined by measuring the Tm of an oligonucleotide/target pair, which is the temperature at which the oligonucleotide and target dissociate; dissociation is detected spectrophotometrically. The higher the Tm, the greater the affinity of the oligonucleotide for the target.

In one embodiment, the region of the oligonucleotide which is modified to increase raf mRNA binding affinity comprises at least one nucleotide modified at the 2′ position of the sugar, e.g., a 2′-O-alkyl, 2′-O-alkyl-O-alkyl or 2′-fluoro-modified nucleotide. Such modifications are routinely incorporated into oligonucleotides and these oligonucleotides have been shown to have a higher Tm (i.e., higher target binding affinity) than 2′-deoxyoligonucleotides against a given target. The effect of such increased affinity is to greatly enhance antisense oligonucleotide inhibition of raf gene expression. RNAse H is a cellular endonuclease that cleaves the RNA strand of RNA:DNA duplexes; activation of this enzyme therefore results in cleavage of the RNA target, and thus can greatly enhance the efficiency of antisense inhibition. Cleavage of the RNA target can be routinely demonstrated by gel electrophoresis.

In another embodiment, the chimeric oligonucleotide is also modified to enhance nuclease resistance. Cells contain a variety of exo- and endo-nucleases which can degrade nucleic acids. A number of nucleotide and nucleoside modifications have been shown to make the oligonucleotide into which they are incorporated more resistant to nuclease digestion than the native oligodeoxynucleotide. Nuclease resistance is routinely measured by incubating oligonucleotides with cellular extracts or isolated nuclease solutions and measuring the extent of intact oligonucleotide remaining over time, usually by gel electrophoresis. Oligonucleotides which have been modified to enhance their nuclease resistance survive intact for a longer time than unmodified oligonucleotides. A variety of oligonucleotide modifications have been demonstrated to enhance or confer nuclease resistance. In one embodiment, the oligonucleotide contains at least one phosphorothioate modification. In one embodiment, the oligonucleotide is fully phosphorothioate modified. In some cases, oligonucleotide modifications which enhance target binding affinity are also, independently, able to enhance nuclease resistance.

In particular embodiments, the oligonucleotides in accordance with this invention are from about 8 to about 50 nucleotides in length. In the context of this invention it is understood that this encompasses non-naturally occurring oligomers as herein before described, having 8 to 50 monomers. In further embodiments, the oligonucleotides comprise from about 8 to about 30 nucleobases (i.e., from about 8 to about 30 linked nucleosides).

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric structure can be further joined to form a circular structure; however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Modified oligonucleotide backbones include, for example, phosphorothioates, chiral phosphorothioates, phosphorodithioates, phosphotriesters, aminoalkylphosphotri-esters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Certain modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In other oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262. Further teaching of PNA compounds can be found in Nielsen et al. (Science, 1991, 254, 1497-1500).

Certain embodiments of the invention are oligonucleotides with phosphorothioate backbones and oligonucleosides with heteroatom backbones, and in particular —CH²—NH—O—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Other embodiments are oligonucleotides having morpholino backbone structures of the above-referenced U.S. Pat. No. 5,034,506.

Modified raf antisense oligonucleotides may contain one or more substituted sugar moieties. Preferred oligonucleotides comprise one of the following at the 2′ position: OH; F; O-, S-, or N-alkyl, O-alkyl-O-alkyl, O-, S-, or N-alkenyl, or O-, S- or N-alkynyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)₂ON(CH₃)₂, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other oligonucleotides comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

One modification is 2′-methoxyethoxy (2′-O—CH₂CH₂OCH₃, also known as 2′-O-(2-methoxyethyl) or 2′-MOE) (Martin et al., Helv. Chim. Acta 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, and 2′-dimethylaminoethoxyethoxy (2′-DMAEOE). Other modifications include 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide.

Oligonucleotides may also have sugar mimetics such as cyclobutyl moieties in place of the pentofuranosyl sugar. Representative United States patents that teach the preparation of such modified sugars structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C or m5c), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in the Concise Encyclopedia Of Polymer Science And Engineering 1990, pages 858-859, Kroschwitz, J. I., ed. John Wiley & Sons, those disclosed by Englisch et al. (Angewandte Chemie, International Edition 1991, 30, 613-722), and those disclosed by Sanghvi, Y. S., Chapter 15, Antisense Research and Applications 1993, pages 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2.degree. C. (Sanghvi, Y. S., Crooke, S. T. and Lebleu, B., eds., Antisense Research and Applications 1993, CRC Press, Boca Raton, pages 276-278). In certain embodiments, 5-methylcytosine substitutions are present in combination with 2′-O-methoxyethyl sugar modifications.

Representative United States patents that teach the preparation of certain of the above noted modified nucleobases as well as other modified nucleobases include, but are not limited to, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941.

In certain embodiments, an oligonucleotide of the invention comprises a cap structure” or “terminal cap moiety.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA 1989, 86, 6553-6556), cholic acid (Manoharan et al., Bioorg. Med. Chem. Lett. 1994, 4, 1053-1059), a thioether, e.g., hexyl-5-tritylthiol (Manoharan et al., Ann. N.Y. Acad. Sci. 1992, 660, 306-309; Manoharan et al., Bioorg. Med. Chem. Let. 1993, 3, 2765-2770), a thiocholesterol (Oberhauser et al., Nucl. Acids Res. 1992, 20, 533-538), an aliphatic chain, e.g., dodecandiol or undecyl residues (Saison-Behmoaras et al., EMBO J. 1991, 10, 1111-1118; Kabanov et al., FEBS Lett. 1990, 259, 327-330; Svinarchuk et al., Biochimie 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett. 1995, 36, 3651-3654; Shea et al., Nucl. Acids Res. 1990, 18, 3777-3783), a polyamine or a polyethylene glycol chain (Manoharan et al., Nucleosides & Nucleotides 1995, 14, 969-973), or adamantane acetic acid (Manoharan et al., Tetrahedron Lett. 1995, 36, 3651-3654), a palmityl moiety (Mishra et al., Biochim. Biophys. Acta 1995, 1264, 229-237), or an octadecylamine or hexylamino-carbonyl-oxycholesterol moiety (Crooke et al., J. Pharmacol. Exp. Ther., 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

The oligonucleotides used in accordance with this invention may be conveniently and routinely made through the well-known technique of solid phase synthesis. Equipment for such synthesis is sold by several vendors including Applied Biosystems. Any other means for such synthesis may also be employed; the actual synthesis of the oligonucleotides is well within the talents of the routineer. It is also well known to use similar techniques to prepare other oligonucleotides such as the phosphorothioates and alkylated derivatives. It is also well known to use similar techniques and commercially available modified amidites and controlled-pore glass (CPG) products such as biotin, fluorescein, acridine or psoralen-modified amidites and/or CPG (available from Glen Research, Sterling, Va.) to synthesize fluorescently labeled, biotinylated or other modified oligonucleotides such as cholesterol-modified oligonucleotides.

The raf antisense oligonucleotides of the present invention modulate raf gene expression. In certain embodiments, they inhibit c-raf gene expression. The sequences of exemplary c-raf antisense oligonucleotides that may be used according to the methods and compositions of the present invention are shown in Tables 1-4. The antisense oligonucleotides listed in the tables target SEQ ID NO: 29 (GENBANK Accession No. NM_{—}002880). ‘Target start position’ indicates the 5′-most nucleotide to which the antisense oligonucleotide is targeted. ‘Target stop position’ indicates the 3′-most nucleotide to which the antisense oligonucleotide is targeted.

TABLE 1
Human c-raf Kinase Antisense Oligonucleotides

| Target | Target | | | SEQ |
|---|---|---|---|---|
| Start | Stop | | | ID |
| position | position | OligoSeq | Site | NO |
| 287 | 306 | CGGGAGGCGGTCACATTCGG | 5′UTR | 23 |
| 297 | 316 | GGTGAGGGAGCGGGAGGCGG | 5′UTR | 16 |
| 314 | 333 | TCCTCCTCCCCGCGGCGGGT | 5′UTR | 20 |
| 317 | 336 | CGCTCCTCCTCCCCGCGGCG | 5′UTR | 17 |
| 323 | 342 | CTCGCCCGCTCCTCCTCCCC | 5′UTR | 21 |
| 337 | 356 | TTCGGCGGCAGCTTCTCGCC | 5′UTR | 18 |
| 357 | 376 | GCCGCCCCAACGTCCTGTCG | 5′UTR | 19 |
| 380 | 399 | ATTCTTAAACCTGAGGGAGC | 5′UTR | 5 |
| 395 | 414 | GATGCAGCTTAAACAATTCT | 5′UTR | 6 |
| 403 | 422 | GCTCCATTGATGCAGCTTAA | AUG | 2 |
| 413 | 432 | CCCTGTATGTGCTCCATTGA | AUG | 3 |
| 483 | 494 | TGAAGGTGAGCTGGAGCCAT | Coding | 1 |
| 483 | 502 | AGAGATGCAGCTGGAGCCAT | Coding | 10 |
| 483 | 493 | AGGTGAAGGCCTGGAGCCAT | Coding | 11 |
| 2356 | 2375 | GGTGCAAAGTCAACTAGAAG | STOP | 4 |
| 2391 | 2410 | CTGGCTTCTCCTCCTCCCCT | 3′UTR | 22 |
| 2551 | 2570 | CAGCACTGCAAATGGCTTCC | 3′UTR | 7 |
| 2711 | 2730 | TCAGGGCTGGACTGCCTGCT | 3′UTR | 15 |
| 2741 | 2760 | CTGATTTCCAAAATCCCATG | 3′UTR | 13 |
| 2771 | 2790 | TCCCGCCTGTGACATGCATT | 3′UTR | 8 |
| 2800 | 2819 | TCTGGCGCTGCACCACTCTC | 3′UTR | 24 |
| 2801 | 2820 | GTCTGGCGCTGCACCACTCT | 3′UTR | 12 |
| 2831 | 2850 | CTGGGCTGTTTGGTGCCTTA | 3′UTR | 14 |
| 2964 | 2983 | GCCGAGTGCCTTGCCTGGAA | 3′UTR | 9 |

Exemplary 2′-modified c-raf antisense oligonucleotides comprising either phosphodiester (P═O) or phosphorothioate (P═S) backbones and uniformly substituted at the 2′ position of the sugar with either a 2′-O-methyl, 2′-O-propyl, or 2′-fluoro group are shown in Table 2.

TABLE 2
Uniformly 2′ Sugar-modified c-raf Oligonucleotides

| Target | Target | | | | |
|---|---|---|---|---|---|
| Start | Stop | | | | SEQ |
| posi- | posi- | | | | ID |
| tion | tion | OligoSeq | Site | Motif | NO. |
| 287 | 306 | CGGGAGGCGGTCACATTCGG | 5′UTR | OMe/P = S | 23 |
| 287 | 306 | CGGGAGGCGGTCACATTCGG | 5′UTR | OPr/P = O | 23 |
| 287 | 306 | CGGGAGGCGGTCACATTCGG | 5′UTR | 2′F/P = S | 23 |
| 297 | 316 | GGTGAGGGAGCGGGAGGCGG | 5′UTR | OMe/P = S | 16 |
| 297 | 316 | GGTGAGGGAGCGGGAGGCGG | 5′UTR | OPr/P = O | 16 |
| 317 | 336 | CGCTCCTCCTCCCCGCGGCG | 5′UTR | OMe/P = S | 17 |
| 317 | 336 | CGCTCCTCCTCCCCGCGGCG | 5′UTR | OPr/P = O | 17 |
| 337 | 356 | TTCGGCGGCAGCTTCTCGCC | 5′UTR | OMe/P = S | 18 |
| 337 | 356 | TTCGGCGGCAGCTTCTCGCC | 5′UTR | OPr/P = O | 18 |
| 357 | 376 | GCCGCCCCAACGTCCTGTCG | 5′UTR | OMe/P = S | 19 |
| 357 | 376 | GCCGCCCCAACGTCCTGTCG | 5′UTR | OPr/P = O | 19 |
| 380 | 399 | ATTCTTAAACCTGAGGGAGC | 5′UTR | OMe/P = S | 5 |
| 380 | 399 | ATTCTTAAACCTGAGGGAGC | 5′UTR | OPr/P = O | 5 |
| 395 | 414 | GATGCAGCTTAAACAATTCT | 5′UTR | OMe/P = S | 6 |
| 395 | 414 | GATGCAGCTTAAACAATTCT | 5′UTR | OPr/P = O | 6 |
| 403 | 422 | GCTCCATTGATGCAGCTTAA | AUG | OMe/P = S | 2 |
| 403 | 422 | GCTCCATTGATGCAGCTTAA | AUG | OPr/P = O | 2 |
| 413 | 432 | CCCTGTATGTGCTCCATTGA | AUG | OMe/P = S | 3 |
| 413 | 432 | CCCTGTATGTGCTCCATTGA | AUG | OPr/P = O | 3 |
| 2771 | 2790 | TCCCGCCTGTGACATGCATT | 3′UTR | OMe/P = S | 8 |

Exemplary chimeric oligonucleotides having SEQ ID NO: 8 and having central “gap” regions of 6, 8, or 10 deoxynucleotides flanked by two regions of 2′-O-methyl modified nucleotides are shown in Table 3. Backbones may be uniformly phosphorothioate. Additional chimeric oligonucleotides having one or more regions of 2′-O-methyl modification and uniform phosphorothioate backbones are shown in Table 3. All are phosphorothioates; bold regions indicate 2′-O-methyl modified regions.

TABLE 3
Chimeric 2′-O-methyl P = S c-raf
oligonucleotides

| Target | Target | | | SEQ |
|---|---|---|---|---|
| Start | Stop | | | ID |
| position | position | OligoSeq | Site | NO. |
| 314 | 333 | TCCTCCTCCCCGCGGCGGGT | 5′UTR | 20 |
| 314 | 333 | TCCTCCTCCCCGCGGCGGGT | 5′UTR | 20 |
| 323 | 342 | CTCGCCCGCTCCTCCTCCCC | 5′UTR | 21 |
| 323 | 342 | CTCGCCCGCTCCTCCTCCCC | 5′UTR | 21 |
| 325 | 344 | TTCTCGCCCGCTCCTCCTCC | 5′UTR | 25 |
| 325 | 344 | TTCTCGCCCGCTCCTCCTCC | 5′UTR | 25 |
| 2386 | 2405 | TTCTCCTCCTCCCCTGGCAG | 3′UTR | 26 |
| 2391 | 2410 | CTGGCTTCTCCTCCTCCCCT | 3′UTR | 22 |
| 2391 | 2410 | CTGGCTTCTCCTCCTCCCCT | 3′UTR | 22 |
| 2395 | 2414 | CCTGCTGGCTTCTCCTCCTC | 3′UTR | 27 |

Additional exemplary chimeric oligonucleotides with various 2′ modifications are shown in Table 4. All are phosphorothioates; bold regions indicate 2′-modified regions.

TABLE 4
Chimeric 2′-modified P = S c-raf oligonucleotides

| Target | Target | | | | |
|---|---|---|---|---|---|
| Start | Stop | | | | SEQ ID |
| position | position | OligoSeq | Site | Modification | NO. |
| 2771 | 2790 | TCCCGCCTGTGACATGCATT | 3′UTR | 2′-o-Me | 8 |
| 2771 | 2790 | TCCCGCCTGTGACATGCATT | 3′UTR | 2′-O-Me | 8 |
| 2771 | 2790 | TCCCGCCTGTGACATGCATT | 3′UTR | 2′-O-Me | 8 |
| 2771 | 2790 | TCCCGCCTGTGACATGCATT | 3′UTR | 2′-O-Pro | 8 |
| 2771 | 2790 | TCCCGCCTGTGACATGCATT | 3′UTR | 2′-F | 8 |
| 2800 | 2819 | TCTGGCGCTGCACCACTCTC | 3′UTR | 2′-O-Me | 24 |
| 2800 | 2819 | TCTGGCGCTGCACCACTCTC | 3′UTR | 2′-F | 24 |

Exemplary chimeric oligonucleotides with 2′-O-propyl sugar modifications and chimeric P═O/P═S backbones are shown in Table 5, in which italic regions indicate regions which are both 2′-modified and have phosphodiester backbones.

TABLE 5
Chimeric 2′-modified P = S/P = O c-raf oligonucleotides

| Target | Target | | | | SEQ |
|---|---|---|---|---|---|
| Start | Stop | | | | ID |
| position | position | OligoSeq | Site | Modification | NO. |
| 2771 | 2790 | TCCCGCCTGTGACATGCATT | 3′UTR | 2′-O-Pro | 8 |
| 2800 | 2819 | TCTGGCGCTGCACCACTCTC | 3′UTR | 2′-O-Pro | 24 |

It is understood that for any of the oligonucleotides described herein may include one or more uridine residues instead of one or more thymidine residues. In addition, 2′-methoxyethyl-5-methyluridine (2′MOE^{Me}U) nucleosides are also sometimes designated as 2′-methoxyethylribothymidine (2′-MOE T).

In particular embodiments, modified oligonucleotides of the present invention comprise a gapmer or gap-widened structure. In certain embodiments, a modified oligonucleotide of the present invention comprises: a gap segment consisting of linked deoxynucleosides; a 5′ wing segment consisting of linked nucleosides; and a 3′ wing segment consisting of linked nucleosides; wherein the gap segment is positioned between the 5′ wing segment and the 3′ wing segment, and wherein each nucleoside of each wing segment comprises a modified sugar. In one embodiment, the modified oligonucleotide consists of 20 nucleobases. In a particular embodiment, the modified oligonucleotide comprises: a gap segment consisting of eight linked deoxynucleosides; a 5′ wing segment consisting of six linked nucleosides; and a 3′ wing segment consisting of six linked nucleosides; wherein the gap segment is positioned between the 5′ wing segment and the 3′ wing segment; wherein each nucleoside of each wing segment comprises a 2′-O-methoxyethyl sugar; wherein each internucleoside linkage is a phosphorothioate linkage; and wherein each cytosine is a 5-methylcytosine.

In particular embodiments, methods of the present invention employ a modified oligonucleotide 100% complementary to nucleobases 2771 to 2790 of SEQ ID NO:29, a human c-raf mRNA. In particular embodiments, the nucleobase sequence of the oligonucleotide comprises or consists of the nucleobase sequence provided in SEQ ID NO:28.

In one embodiment, a c-raf antisense oligonucleotide is a full phosphorothioate analog consisting of the sequence, TCCCGCCTGTGACATGCATT (SEQ ID NO:8), with 2′-O-methoxyethyl substitutions at positions 1-6 and 15-20, and wherein residues 7-14 are unmodified 2′-deoxy. In a highly related embodiment, a c-raf antisense oligonucleotide is a full phosphorothioate analog consisting of the sequence, UCCCGCCTGTGACAUGCAUU (SEQ ID NO:28), with 2′-O-methoxyethyl substitutions at positions 1-6 and 15-20, and wherein residues 7-14 are unmodified 2′-deoxy, which may also be depicted as 5′-^{Me}U^{Me}C^{Me}C^{Me}CG^{Me}CCTGTGACA^{Me}UG^{Me}CA^{Me}U^{Me}U-3′ (SEQ ID NO:28), wherein the underlined residues are 2′MOE nucleosides.

The present invention further includes salt forms of the raf antisense oligonucleotides, such as, e.g., nonadecasodium salts. In one embodiment, the raf antisense oligonucleotide is a nonadecasodium salt of a c-raf antisense oligonucleotide that is a full phosphorothioate analog consisting of the sequence, UCCCGCCTGTGACAUGCAUU (SEQ ID NO:28), with 2′-O-methoxyethyl substitutions at positions 1-6 and 15-20, and wherein residues 7-14 are unmodified 2′-deoxy, which has a molecular formula of C₂₃₄H₃₁₀N₆₈O₁₂₆P₁₉S₁₉Na₁₉.

The above oligonucleotides may be prepared by methods known in the art. In one embodiment, they are synthesized by a multi-step process that may be divided into two distinct operations: solid phase synthesis and downstream processing. In the solid-phase synthesis operation, the nucleoside sequence is assembled by a computer controlled solid-phase synthesizer. The downstream process includes deprotection, reverse-phase chromatographic purification, isolation, and lyophilization.

The present invention contemplates the use of any and all raf antisense oligonucleotides comprising or consisting of one or more of these exemplified sequences, in addition to any other raf antisense oligonucleotide capable of modulating raf gene expression. In particular embodiments, a raf antisense oligonucleotide of the present invention selectively modulates expression of one raf gene, while in other embodiments, a raf antisense oligonucleotide of the present invention modulates expression of two or more raf genes.

### D. PHARMACEUTICAL COMPOSITIONS

The present invention further provides pharmaceutical compositions and formulations comprising one or more raf antisense oligonucleotides, alone or in combination with one or more additional therapeutic agent, e.g., an ocular therapeutic agent, including but not limited to any of the raf antisense oligonucleotides and additional therapeutic agents described herein.

Raf antisense oligonucleotides, alone or in combination with one or more additional therapeutic agents, may be formulated as pharmaceutical compositions suitable for delivery to a subject, e.g., a patient diagnosed with or considered at risk of developing an ocular disorder, e.g., a macular edema. The pharmaceutical compositions of the invention will often further comprise one or more diluents, buffers (e.g., neutral buffered saline or phosphate buffered saline), carbohydrates (e.g., glucose, mannose, sucrose, dextrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

Suitable formulations for use in the present invention can be found, e.g., in Remington's Pharmaceutical Sciences, Mack Publishing Company, Philadelphia, Pa., 17^{th} Ed. (1985). Often, pharmaceutical compositions will comprise a solution of the therapeutic agent, e.g., raf antisense oligonucleotide, in an acceptable carrier, such as an aqueous carrier. Any of a variety of aqueous carriers can be used, e.g., water, buffered water, 0.4% saline, 0.9% isotonic saline, 0.3% glycine, 5% dextrose, and the like, and may include glycoproteins for enhanced stability, such as albumin, lipoprotein, globulin, etc. Often, normal buffered saline (135-150 mM NaCl) or 5% dextrose will be used. These compositions can be sterilized by conventional sterilization techniques, such as filtration. The resulting aqueous solutions may be packaged for use or filtered under aseptic conditions and lyophilized, the lyophilized preparation being combined with a sterile aqueous solution prior to administration. The compositions may also contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, etc.

In addition to such pharmaceutical carriers, cationic lipids may be included in the formulations to facilitate oligonucleotide uptake. One such composition shown to facilitate uptake is LIPOFECTIN (a 1:1 liposome formulation of the cationic lipid N-[1-(2,3-dioleyloxy)propyl]-N,N,N-trimethylammonium chloride (DOTMA) and dioleoyl phosphatidylethanolamine (DOPE)) (BRL, Bethesda Md.).

In particular embodiments, pharmaceutical compositions of the present invention are formulated for ocular injection, e.g., intravitreal injection. For example, one pharmaceutical composition of the present invention comprises a sterile solution for injection containing a buffered (about pH 7.4) solution of a raf antisense oligonucleotide in water. In certain embodiments, the buffer is a phosphate or citrate buffer. In a particular embodiment, the oligonucleotide is a c-raf antisense oligonucleotide that is a full phosphorothioate analog consisting of the sequence, UCCCGCCTGTGACAUGCAUU (SEQ ID NO:28), with 2′-O-methoxyethyl substitutions at positions 1-6 and 15-20, and wherein residues 7-14 are unmodified 2′-deoxy. In particular embodiments, the oligonucleotide is present at a concentration of about 10 mg/mL, 1-100 mg/mL, 2-50 mg/mL, 2-25 mg/mL, or 5-20 mg/mL.

In one particular embodiment, the present invention includes a unit dosage form of a pharmaceutical composition comprising or consisting of: about 10 mg or 10 mg of a nonadecasodium salt of a c-raf antisense oligonucleotide that is a full phosphorothioate analog consisting of the sequence, UCCCGCCTGTGACAUGCAUU (SEQ ID NO:28), with 2′-O-methoxyethyl substitutions at positions 1-6 and 15-20, and wherein residues 7-14 are unmodified 2′-deoxy; 0.04 mg of sodium phosphate monobasic monohydrate; 0.42 mg of sodium phosphate dibasic heptahydrate; 8.3 mg of sodium chloride; sodium hydroxide as required for pH adjustment to 7.4; and water to a total volume of 1.0 mL. The present invention further includes unit dosage forms of a pharmaceutical composition comprising or consisting of: 1 mg to 50 mg, 1 mg to 25 mg, 2 mg to 25 mg, 5 mg to 20 mg, or 5 mg to 15 mg of the c-raf antisense oligonucleotide described immediately above, or any other raf antisense oligonucleotide described herein.

Pharmaceutical compositions of the present invention may be packaged in suitable unit dosage forms. For example, in certain embodiments, the present invention includes a vial comprising about 1 ml of a pharmaceutical composition of the present invention. In other embodiments, the present invention includes a vial comprising 1 ml to 10 ml, 1 ml to 5 ml, about 2 ml, about 3 ml, about 4 ml, or about 5 ml of a pharmaceutical composition of the present invention. The vial may be a clear glass vial and may further include a stopped and aluminum overseal.

Pharmaceutical compositions of the present invention may optionally be stored at refrigerated at 2-8° C.+/−5° C. (e.g., 4-8° C.) conditions, protected from light.

The present invention further includes kits comprising one or more pharmaceutical compositions of the present invention. In particular embodiments, a kit of the present invention further comprises a pharmaceutical composition comprising an additional therapeutic agent, such as an ocular therapeutic agent described above.

### EXAMPLES

### Example 1

### Ocular Tissue Distribution and Clearance of C-Raf Antisense Oligonucleotides from Retina-Choroid Following Intravitreal Administration

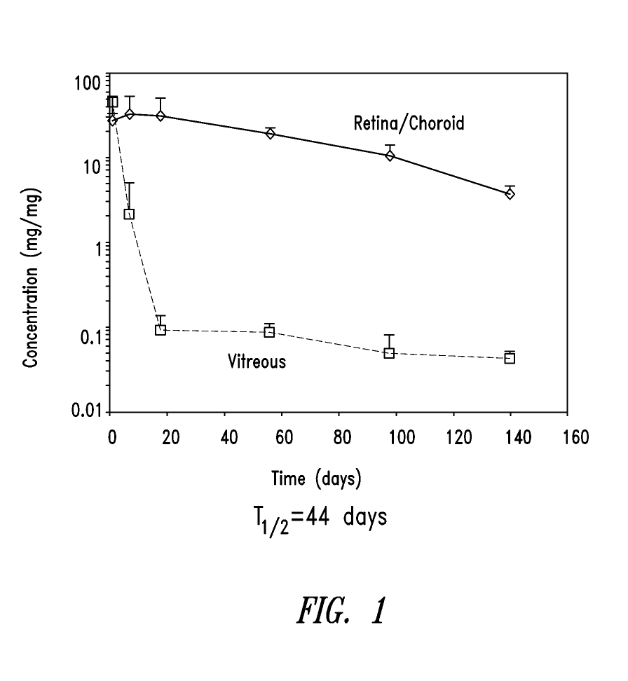

The clearance of a c-raf antisense oligonucleotide from the retina-choroid was determined following a single intravitreal injection into rabbits of 90 μg of the nonadecasodium salt of a c-raf antisense oligonucleotide that is a full phosphorothioate analog consisting of the sequence, UCCCGCCTGTGACAUGCAUU (SEQ ID NO:28), with 2′-O-methoxyethyl substitutions at positions 1-6 and 15-20, and wherein residues 7-14 are unmodified 2′-deoxy nucleotides (the “2′MOE oligonucleotide”). The concentration of the 2′MOE oligonucleotide in the vitreous and the retina/choroid was determined at various time points following administration. As shown in FIG. 1, clearance from the vitreous was rapid, while clearance from the retina/choroid was slow, with the T_{1/2} for the retina/choroid being 44 days. The concentration of the 2′MOE oligonucleotide in the vitreous at day 8 was 1.3±1.4 μg/ml, and concentrations in the vitreous at later time points were below the limit of quantitation. Peak concentrations in the retina-choroid occurred 7 days after injection, with the level being 32.2±21.6 μg/g. After day 8, concentrations declined slowly with 3.8±0.7 μg/g present 140 days post injection. This pattern of drug concentrations following a single intravitreal injection indicated that absorption into the retina-choroid occurred over several days, which is consistent with the presence of oligonucleotide in the vitreous.

Clearance and accumulation of the 2′MOE oligonucleotide in the rabbit retina/choroid was also determined following administration of three doses of 90 μg, with each dose being administered 4 to 6 weeks apart. As shown in Table 6, every 4 to 6 week dosing led to a 2.5-fold increase in concentration at the end of all three doses, as compared to rabbits treated with a single dose.

TABLE 6
Tissue concentration of 2′MOE oligonucleotide.

| Dose (μg) | Single (μg/g) | Multiple × 3 (μg/g) |
|---|---|---|
| 90 | 32.2. ± 21.6 | 81 ± 37 |

The ocular tissue distribution of the 2′MOE oligonucleotide in rabbit eyes was determined by performing immunolocalization experiments. 2′MOE oligonucleotide was detected in all layers of the retina, including the outer and inner plexiform layers, the inner nuclear layer, the outer limiting membrane, and ganglion cells. 2′MOE oligonucleotide was also detected in the ciliary body, choroidal tissue (RPE cells), and the optic nerve.

Proinflammatory effects in the eyes are a tolerability issue for many phosphorothioate oligonucleotides, so the inflammatory effects of the 2′MOE oligonucleotide was compared to the inflammatory effects of another phosphorothioate oligonucleotide, which did not include 2′-MOE modifications (control oligonucleotide). No ocular inflammation, i.e., cyclitis or vitritis, was detected in rabbit eyes treated with a single dose of the 2′MOE oligonucleotide (Table 7). In contrast, the phosphorothioate oligonucleotide lacking the 2′-MOE modifications showed significant proinflammatory effects.

TABLE 7
Incidence of vitritis in rabbit eyes treated with a single
dose of oligonucleotide

| | Control oligonucleotide | 2′MOE |
|---|---|---|
| Dose (μg) | Vitravene ™ | oligonucleotide |
| 3 | 11/12 | 0/12 |
| 90 | 11/12 | 0/12 |

### Example 2

### Ocular Tissue Distribution and Systemic Exposure Following Intravitreal Administration of C-Raf Antisense Oligonucleotides

To determine the ocular tissue distribution and systemic exposure of animals following intravitreal administration of the 2′MOE oligonucleotide described in the preceding example, the 2′MOE oligonucleotide was administered to monkeys for six months, and its concentration in plasma, the eye, and various other tissues was determined. This study was conducted using 10 male and 10 female moneys. The 2′MOE oligonucleotide was administered via a single (Group 5) or repeated (Groups 1-4 and 6) intravitreal injections inserted through the pars plana using a 29 gauge needle and a 50 μl injection volume. Group 1 control animals received no 2′MOE oligonucleotide; Group 2 animals received doses of 125 μg of 2′MOE oligonucleotide; Group 3 animals received doses of 250 μg; Group 4 animals received doses of 500 μg; Group 5 animals received a dose of 125 μg; and Group 6 animals received doses of 125 μg. Doses were administered on day 1 for all groups, day 176 for groups 1-4, and days 90 and 176 for group 6.

Blood samples were collected pre-dose and at approximately 6, 24, 48, and 72 hours post-dose on Day 176 for animals in Groups 2 to 4, and 2′MOE oligonucleotide plasma levels were determined. At sacrifice, both eye tissues/fluid and systemic tissues were collected from all the animals for oligonucleotide concentration analysis. Systemic tissues that were collected included kidney cortex, liver, spleen, and ovaries/testes. Concentrations of the 2′MOE oligonucleotide in plasma and vitreous was determined using a hybridization-dependent nuclease ELISA assay with fluorescence detection. The limit of quantitation (LOQ) of this assay was 0.00386 μg/ml. Tissue and vitreous samples were analyzed by capillary gel electrophoresis method (CGE) with UV detection. The limit of quantitation (LOQ) in tissues and vitreous were approximately 1.16 μg/ml and 1.16 μg/ml, respectively.

2′MOE oligonucleotide concentrations in plasma were measurable at all time points examined for Groups 2-4 and were in the range of 0.0071 to 0.0338 μg/ml. The measured concentrations increased slightly with dose. Maximum plasma concentration (Cmax) ranged from 0.0227 to 0.036 μg/ml at a dose range of 125 to 500 μg, and Cmax was generally observed at the first sampling time point, 6 hr. 2′MOE oligonucleotide concentrations decreased slightly with time with a mean apparent half-life of 40-80 hr.

Concentrations in vitreous were measured using both CGE and ELISA method. On day 183 (7 days after the last dose administration), concentrations of 2′MOE oligonucleotide measured in the vitreous ranged from 2.42 to 6.97 μg/ml (measured by ELISA method) over the dose range of 125 to 500 μg. Thus, concentrations in the vitreous were dose-dependent and close to dose-proportional. The 2′MOE oligonucleotide cleared slowly from the vitreous, with an estimated half-life of 67 days.

2′MOE oligonucleotide was measured in retina, choroid, and lens at all dose levels tested and up to 112 days after a single dose administration. At equivalent dose levels, highest oligonucleotide concentrations were observed in retina, followed by choroid and lens. For example, concentrations of 2′MOE oligonucleotide were 35.9 μg/g, 20.5 μg/g, and 5.53 μg/g in retina, choroid, and lens, respectively, at the 500 μg dose one week after the second dose. Concentration in retina, choroid and lens increased with dose but was slightly less than dose proportional.

Kinetics of 2′MOE oligonucleotide in the eye was determined following a single 125 μg dose. Maximum concentration (Cmax) was observed at the first sampling time point (28 days after dose administration), and was 24.4, 14.1, 2.99 μg/g and 0.14 μg/ml in retina, choroid, lens, and vitreous, respectively. Total exposure measured as AUC was equivalent in retina and choroid, and was nearly 100-fold higher than the exposure measured in the vitreous. Concentration in the eye tissues were observed to be several orders of magnitude higher than corresponding plasma concentrations. 2′MOE oligonucleotide cleared slowly from the eye, with an estimated elimination half-life of 38 to 89 days dependent upon the tissue type.

The 2′MOE oligonucleotide showed negligible systemic exposure following intravitreal injection. The 2′MOE oligonucleotide was measurable in monkey kidney following administration for six months at all dose levels. Concentrations in the kidney were dose-dependent, increased from 6.17 μg/g to 16.2 μg/g as dose increased from 125 μg to 500 μg, and were dose-proportional at the 250 to 500 μg dose range. At the highest dose level (500 μg), concentrations were also measurable in liver and spleen. Oligonucleotide concentrations in the kidney were 10-fold higher than liver, and nearly 20-fold higher than spleen. No measurable levels were detected in ovaries and a very low level was detected in testes of only one animal at any dose level tested. Concentrations of 2′MOE oligonucleotide in systemic tissues were far smaller than the threshold of toxicity levels.

Based upon these studies, in humans, bilateral injection of 330 μg of the 2′MOE oligonucleotide would be predicted to yield a 0.01 mg/kg systemic dose.

These data indicate that intravitreal injection of 2′MOE oligonucleotide results in greatest exposure to retina and choroid, with minimal exposure to systemic tissues. The clearance from retina and choroid was slow, with a half-life of approximately 38-89 days. These properties are favorable for the local therapeutic application for the treatment of retinal disease, and afford infrequent dose intervals.

### Example 3

### Treatment of Diabetic Macular Edema Using C-Raf Antisense Oligonucleotides

Human phase I clinical trials were performed in a single-dose, open-label, dose-escalation trial using a c-raf antisense oligonucleotide that is a full phosphorothioate analog consisting of the sequence, UCCCGCCTGTGACAUGCAUU (SEQ ID NO:28), with 2′-O-methoxyethyl substitutions at positions 1-6 and 15-20, and wherein residues 7-14 are unmodified 2′-deoxy, to treat four cohorts (Cohorts 1-4) of patients having diffuse diabetic macular edema (DME) and a visual acuity of 60-15 letters. Each cohort included three patients, except for Cohort 4, which included six patients, as indicated in Table 8.

TABLE 8
Human Clinical Trial Cohorts

| | | | | # Patients w/ |
|---|---|---|---|---|
| | | Dosage | # Patients | reduced OCT of >140 |
| Cohort | # Patients | (μg of oligo) | treated | microns at week 24 |
| 1 | 3 | 110 | 3/3 | 1/3 |
| 2 | 3 | 350 | 3/3 | 2/3 |
| 3 | 3 | 700 | 3/3 | 2/3 |
| 4 | 6 | 1000 | 6/6 | 1/6 |

The patients' retinal thickness was >250 microns in the central subfield at baseline. Patients underwent an at least 3 month wash out period prior to enrollment, since numerous patients had previously received treatment with photocoagulation, steroids, or anti-VEGF drugs and were often refractory to those treatments. Patients were then administered a single dose of a nonadecasodium salt of the 2′MOE oligonucleotide by intravitreal injection. The salt form of the oligonucleotide was present in a pharmaceutical composition comprising 10 mg oligonucleotide; 0.04 mg of sodium phosphate monobasic monohydrate; 0.42 mg of sodium phosphate dibasic heptahydrate; 8.3 mg of sodium chloride; sodium hydroxide as required for pH adjustment to 7.4; and water for injection to a total volume of 1.0 mL. Cohort 1 patients received 110 μg of the 2′MOE oligonucleotide; Cohort 2 patients received 350 μg of the 2′MOE oligonucleotide; Cohort 3 patients received 700 μg of the 2′MOE oligonucleotide; and Cohort 4 patients received 1000 μg of the 2′MOE oligonucleotide.

Retinal thickness, visual acuity, and safety were observed over the twenty-four week period following administration of the oligonucleotides for two of the three patients in Cohort 1; three of the three patients in Cohort 2 had visual acuity and safety data at the twenty-four week time point; two of three patients in Cohort 2 had retinal thickness data at the twenty-four week time point; and all three patients in Cohort 3 and five of the six patients in Cohort 4 had retinal thickness, visual acuity and safety data at the twenty-four week time point.

Changes in retinal thickness were measured using optical coherence tomography (OCT; Stratus, Carl Zeiss Meditech Ophthalmic Systems Inc., Dublin, Calif., USA) at 0.4, 1, 2, 4, 8, 12, 18, and 24 weeks following administration of the 2′MOE oligonucleotides. OCT demonstrated a reduction in central retinal thickness of more than 140 microns in five out of seven patients examined at the end of the follow-up as compared to baseline, when taking into account all patients in Cohorts 1, 2, and 3 for whom retinal thickness was determined at week twenty-four. When taking into account all patients in Cohorts 1, 2, 3 and 4 for whom retinal thickness was determined at week twenty-four, OCT demonstrated a reduction in central retinal thickness of more than 140 microns in six out of twelve patients examined: one patient in Cohort 1, two patients in Cohort 2, two patients in Cohort 3, and one patient in Cohort 4. For patients in Cohorts 1, 2 and 3, OCT also demonstrated a reduction in central retinal thickness of more than 600 microns in two out of seven patients at the end of the follow-up period as compared to baseline, with one patient in Cohort 2 and one patient in Cohort 3.

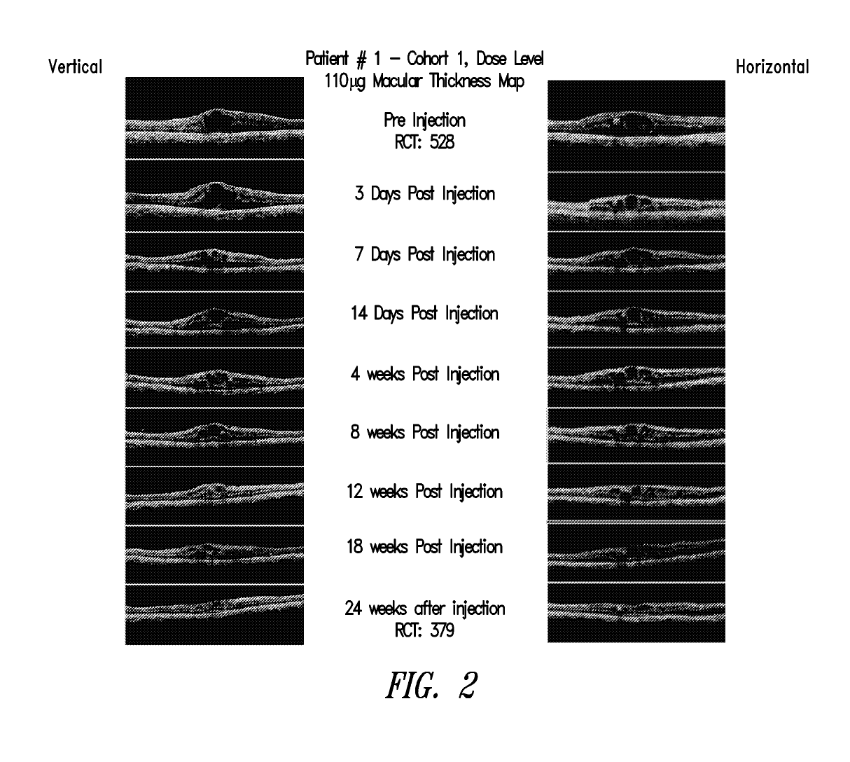

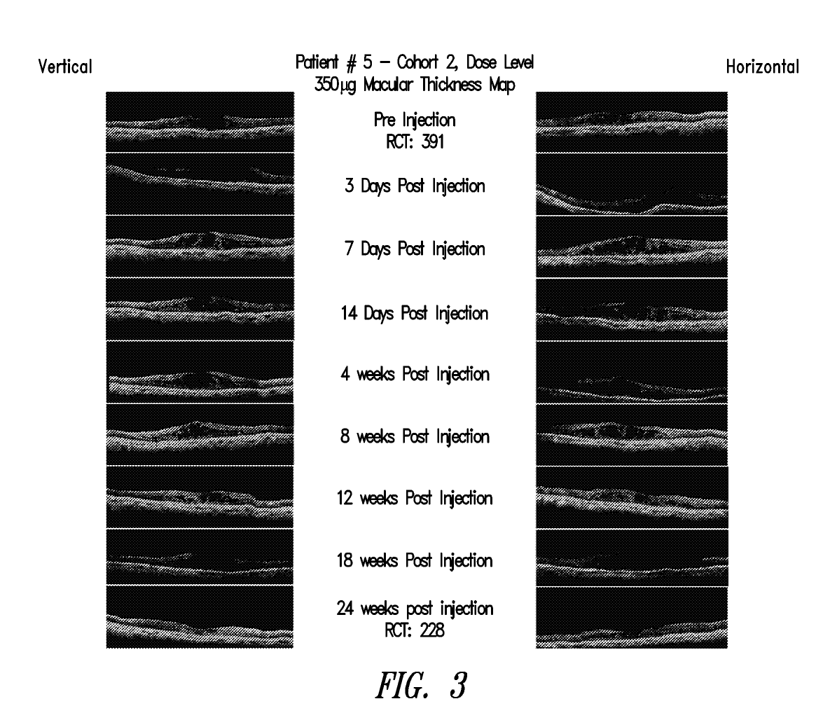

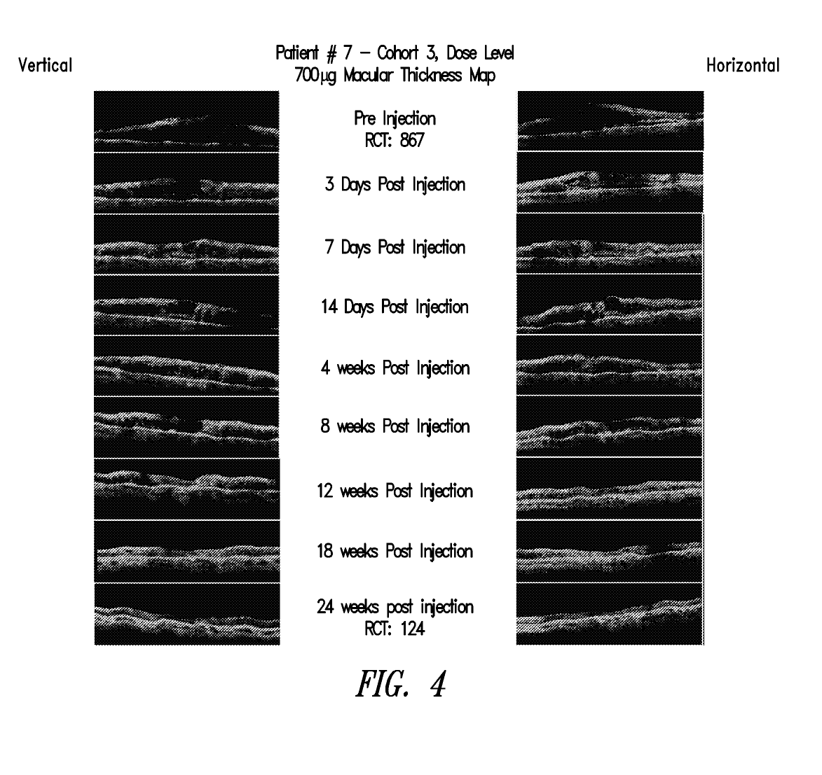

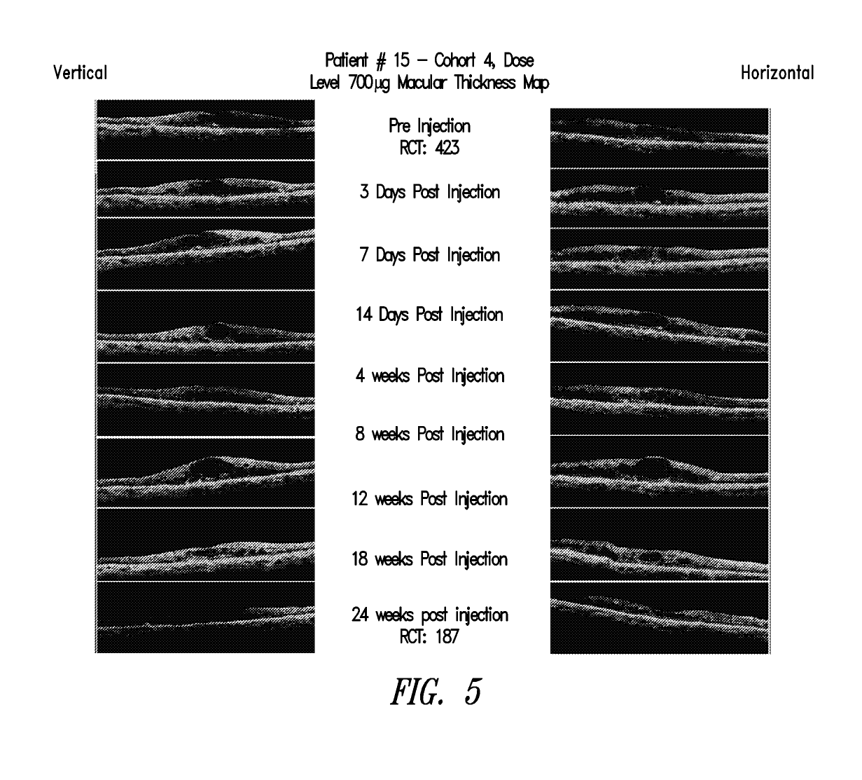

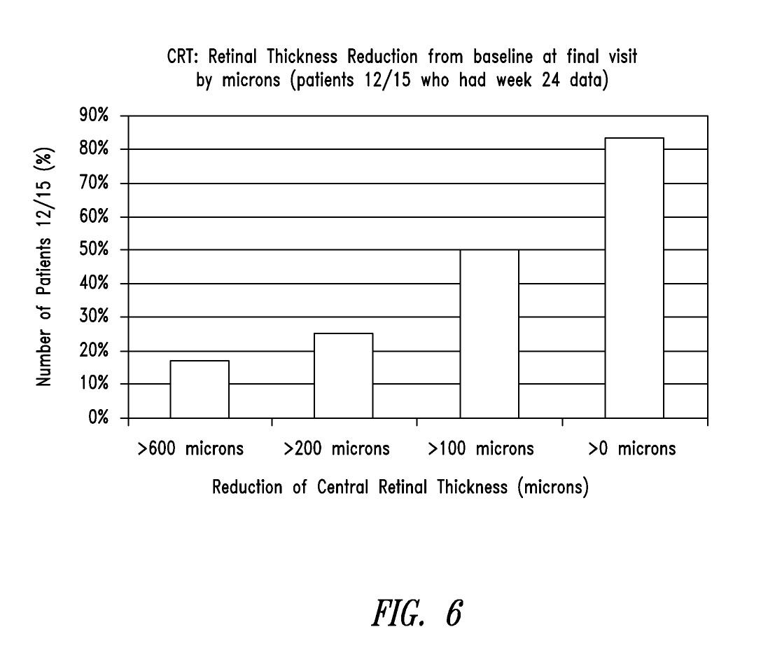

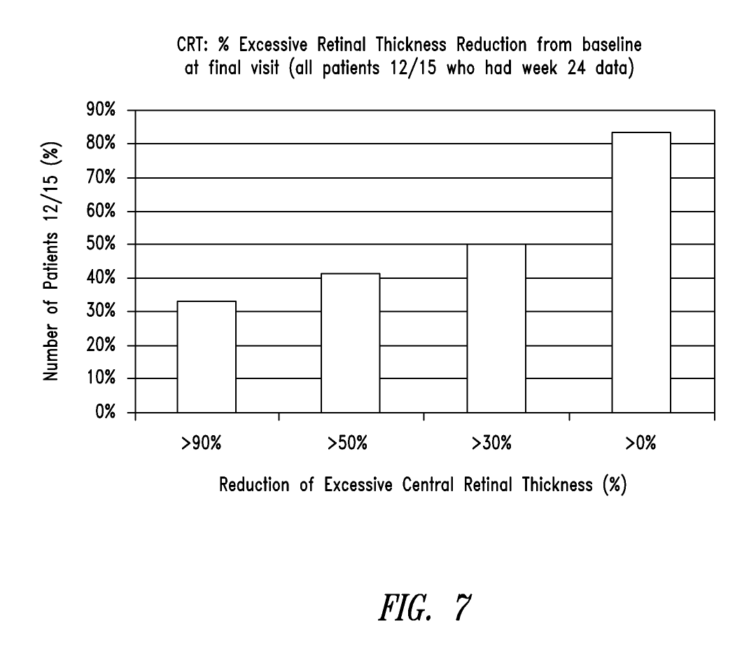

For all patients for whom OCT measurements were taken at twenty-four weeks (12/15 total patients), the mean change in CRT compared to baseline at twenty-four weeks was −169 microns and the mean change (%) in reduction of excess retinal thickness compared to baseline in patients who had OCT performed was −40.5%. The greatest reduction in retinal thickness was observed twenty-four weeks (six months) after the single injection. The results observed in a selected patient from Cohorts 1-4 are shown in FIGS. 2-5, respectively, which provide macular thickness maps showing a reduction in retinal thickness over 24 weeks post-injection. The results in all patients tested are summarized in FIGS. 6 and 7, which show retinal thickness reduction from baseline and % excessive retinal thickness reduction from baseline at twenty-four weeks, respectively.

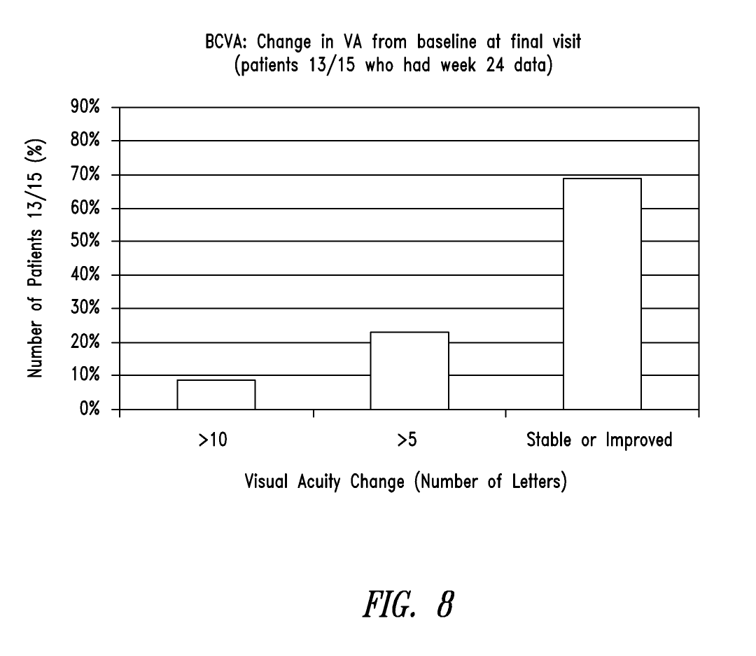

Visual acuity tests were performed, and three patients out of eight gained five or more letters at the end of the follow-up as compared to baseline, when taking into account all patients who completed the week 24 visit: two in Cohort 1, three in Cohort 2 and three patients in Cohort 3. One Cohort 1 patient gained five letters; one Cohort 2 patient gained six letters, and one Cohort 3 patient gained twelve letters. One patient out of eight gained three letters at the end of the twenty-four week follow up compared to baseline, when taking into account all patients in Cohort 1, Cohort 2 and Cohort 3. One patient out of eight had no change in vision at the end of the follow-up as compared to baseline, when taking into account all patients examined at this time in Cohort 1, Cohort 2, and Cohort 3. Three patients out of eight lost vision at the end of the follow-up as compared to baseline, when taking into account all patients examined at this time in Cohort 1, Cohort 2, and Cohort 3, due to either vitreous hemorrhage, development of neovascular glaucoma, or progression of the underlying disease. Of all patients examined at 24 weeks, approximately 69% had stable or improved vision and 23% experienced greater than or equal to 5 letters of visual improvement. This data is summarized in FIG. 8, which shows changes in visual acuity in all patients tested (13/15) at twenty-four weeks. Only four patients showed decrease in BCVA of more than 5 letters at their final visit, likely due to development of opacities in the optic media or progression of the underlying disease.

No drug-related serious adverse effects were observed. Very good safety results were observed even with the highest dose (1,000 μg) tested (data not shown). PK levels of the 2′MOE oligonucleotide remained below the detectable level of 2.00 ng/mL in the patients' blood plasma. A number of patients refractory to other treatments appeared to respond well to treatment with the 2′OME oligonucleotide.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of treating or preventing macular edema, comprising administering to a subject in need thereof a therapeutically effective amount of a composition comprising a modified oligonucleotide 100% complementary to nucleobases 2771 to 2790 of SEQ ID NO:29, wherein the composition is administered no more than once every 90 days.

2. The method of claim 1, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment, and wherein each nucleoside of each wing segment comprises a modified sugar.

3. (canceled)

4. (canceled)

5. The method of claim 1, wherein the oligonucleotide comprises the nucleobase sequence of SEQ ID NO:28.

6. The method of claim 1, wherein the composition is administered no more than once every 180 days.

7. The method of claim 1, wherein the subject is refractory to treatment with one or more of: a steroid, laser treatment, ranibizumab, bevacizumab, and rapamycin.

8. The method of claim 1, wherein at least 100 μg of the oligonucleotide is administered.

9. (canceled)

10. (canceled)

11. A method of reducing or preventing excess retinal thickness, comprising administering to a subject in need thereof a therapeutically effective amount of a composition comprising a modified oligonucleotide 100% complementary to nucleobases 2771 to 2790 of SEQ ID NO:29, wherein the composition is administered no more than once every 90 days.

12. The method of claim 11, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment, and wherein each nucleoside of each wing segment comprises a modified sugar.

13. (canceled)

14. (canceled)

15. The method of claim 11, wherein the oligonucleotide comprises the nucleobase sequence of SEQ ID NO:28.

16. The method of claim 11, wherein the composition is administered no more than once every 180 days.

17. The method of claim 11, wherein the subject is refractory to treatment with one or more of: a steroid, laser treatment, ranibizumab, bevacizumab, and rapamycin.

18. (canceled)

19. (canceled)

20. The method of claim 11, wherein at least 100 μg of the oligonucleotide is administered.

21. (canceled)

22. (canceled)

23. A method of improving visual acuity, comprising administering to a subject in need thereof a therapeutically effective amount of a composition comprising a modified oligonucleotide 100% complementary to nucleobases 2771 to 2790 of SEQ ID NO:29, wherein the composition is administered no more than once every 90 days.

24. The method of claim 23, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment, and wherein each nucleoside of each wing segment comprises a modified sugar.

25. (canceled)

26. (canceled)

27. The method of claim 23, wherein the oligonucleotide comprises the nucleobase sequence of SEQ ID NO:28.

28. The method of claim 23, wherein the composition is administered no more than once every 180 days.

29. The method of claim 23, wherein the subject is refractory to treatment with one or more of: a steroid, laser treatment, ranibizumab, bevacizumab, and rapamycin.

30. (canceled)

31. (canceled)

32. (canceled)

33. (canceled)

34. A method of treating or preventing macular edema, comprising administering to a subject in need thereof a therapeutically effective amount of a composition comprising a modified oligonucleotide 100% complementary to nucleobases 2771 to 2790 of SEQ ID NO:29, wherein at least 100 μg of the oligonucleotide is administered.

35. The method of claim 34, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment, and wherein each nucleoside of each wing segment comprises a modified sugar.

36. (canceled)

37. (canceled)

38. The method of claim 34, wherein the modified oligonucleotide has the nucleobase sequence of SEQ ID NO:28.

39. The method of claim 34, wherein the oligonucleotide is administered no more than once every 90 days.

40. (canceled)

41. (canceled)

42. The method of claim 34, wherein the subject is refractory to treatment with one or more of: a steroid, laser treatment, ranibizumab, bevacizumab, and rapamycin.

43. The method of claim 42, wherein an amount in the range of 100 μg to 1500 μg of the oligonucleotide is administered.

44. A method of treating or preventing macular edema, comprising administering to a subject in need thereof:
(a) a modified oligonucleotide 100% complementary to nucleobases 2771 to 2790 of SEQ ID NO:29; and
(b) one or more of ranibizumab, bevacizumab, and rapamycin,
wherein the oligonucleotide and the one or more of ranibizumab, bevacizumab, and rapamycin are administered in a therapeutically effective amount, and the oligonucleotide is administered no more than once every 90 days.

45.-78. (canceled)
